(19) United States
(12) Patent Application Publication
(10) Pub. No.: US 2020/0323797 A1
(43) Pub. Date: Oct. 15, 2020

(54) Treatment of Obesity-related Conditions

(71) Applicants: INSTITUTO DE MEDICINA MOLECULAR, 1649-028 Lisbon (PT); FUNDAÇÃO CALOUSTE GULBENKIAN, 1067-001 Lisbon (PT)

(72) Inventors: Ana DOMINGOS, 6 2780-156 Oeiras (PT); Gonçalo BERNARDES, 1649-028 Lisbon (PT)

(21) Appl. No.: 16/753,237

(22) PCT Filed: Oct. 8, 2018
(86) PCT No.: PCT/EP2018/077352

§ 371 Date: Apr. 2, 2020

(30) Foreign Application Priority Data
Oct. 6, 2017 (PT) 20171000065945

(51) Int. Cl. A61K 31/137; A61K 47/55; A61K 47/60; A61K 47/64; A61K 47/68; A61P 3/04

(52) U.S. Cl. CPC A61K 31/137; A61K 47/551; A61P 3/04; A61K 47/645; A61K 47/6803; A61K 47/60

(57) ABSTRACT

This invention relates to the finding that the inhibition of solute carrier family 6 member 2 (Slc6a2) exert a sympathomimetic effect outside the brain that promotes weight loss without concomitant hypophagia or hyperkinesia. Compounds for the inhibition of Slc6a2 outside the brain, as well as methods of promoting weight loss and treating obesity using such compounds are provided.

TABLE 1

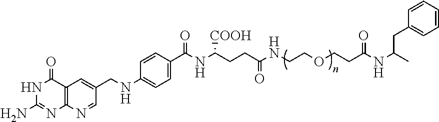

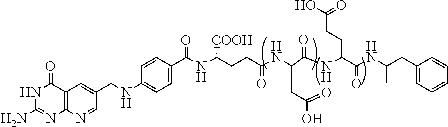

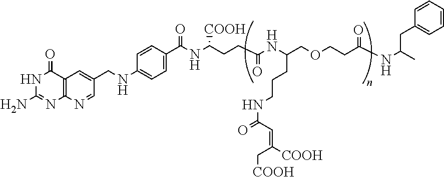

### FIELD

The present invention relates to compounds and methods for the treatment of obesity and related conditions.

### BACKGROUND

Sympathetic innervation of adipose tissue promotes lipolysis and fat mass reduction via norepinephrine (NE) signaling¹. In obesity, chronic local inflammation underlies adipose tissue dysfunction, and macrophages have been shown to play a central role^{1, 2}. The mechanism that links macrophages in white adipose tissue (WAT) to NE remains controversial. Somegroups have reported that anti-inflammatory adipose tissue macrophages (ATMs) in the WAT produce NE to sustain thermogenesisand browning. In direct contradiction, other groups have reported that ATMs do not express a key enzyme required for NE production and that genetic deletion of this enzyme in macrophages has no effect on thermogenesis and body weight.³⁻⁶

Sympathomimetic drugs such as those in the amphetamine (AMPH) class have the highest efficacy among all compounds ever approved as therapeutics for non-monogenic obesity^{7, 8}. The potent anti-obesity effect of AMPH is believed to be mediated by a stimulant action in the brain that supresses appetite and promotes hyperkinesia. AMPH have a preferential biodistribution in the brain rather than in circulation^{9, 10}, and most biological studies focus on its central action in the brain to modulate behaviour¹¹.

Methods for manipulating noradrenergic homeostasis to promote lipolysis and fat mass reduction independently of actions in the brain would be useful in for both therapeutic and cosmetic or well-being purposes.

### SUMMARY

The present inventors have discovered that solute carrier family 6 member 2 (Slc6a2) inhibitors that do not permeate the blood-brain barrier (BBB) exert a sympathomimetic effect outside the brain that promotes weight loss without concomitant hypophagia or hyperkinesia. This may be useful for example in the treatment of obesity and obesity-related conditions.

A first aspect of the invention provides a conjugate comprising a Slc6a2 (norepineophrine transporter NET) inhibitor and a moiety which blocks passage across the blood-brain barrier.

Preferably, the Slc6a2 inhibitor is a norepinephrine reuptake inhibitor, such as amphetamine, a substituted amphetamine, or nisoxetine.

Preferably, the moiety which blocks passage across the blood-brain barrier is a polyether or oligoether or unstructured or structured peptidic units.

Preferred conjugates of the first aspect include PEGylated amphetamine (PEG-AMPH). Suitable conjugates are shown in Table 1.

In some embodiments, the conjugate may be targeted to macrophages, preferably sympathetic neuron-associated macrophages (SAMs), or adipose tissue. For example, a conjugate may further comprise a second moiety which facilitates an affinity to adipose tissue or macrophages, preferably sympathetic neuron-associated macrophages (SAMs). Suitable second moieties include antibodies or folate groups.

A second aspect of the invention provides a conjugate of the first aspect for use as a medicament.

A third aspect of the invention provides a pharmaceutical composition comprising a conjugate of the first aspect and a pharmaceutically acceptable diluent.

A fourth aspect of the invention comprises a method of decreasing fat mass or promoting weight loss comprising administering a Slc6a2 inhibitor that does not cross the BBB, for example a compound of the first aspect or a pharmaceutical composition of the third aspect, to an individual in need thereof.

A method of the fourth aspect may be therapeutic or non-therapeutic (e.g. cosmetic).

A fifth aspect of the invention comprises a method of treatment of obesity comprising administering Slc6a2 inhibitor that does not cross the BBB, for example a conjugate of the first aspect or a pharmaceutical composition of the third aspect, to an individual in need thereof.

A sixth aspect of the invention provides a Slc6a2 inhibitor that does not cross the BBB, a compound of the first aspect or a pharmaceutical composition of the third aspect, for use in a method according to the fourth or fifth aspect.

A seventh aspect of the invention provides the use of a Slc6a2 inhibitor that does not cross the BBB, a conjugate of the first aspect or a pharmaceutical composition of the third aspect, for use in a method according to the fourth or fifth aspect.

Other aspects and embodiments of the invention are described in more detail below.

### BRIEF DESCRIPTION OF THE FIGURES

FIG. 1 shows SAMs import and metabolize norepinephrine via SLC6A2 and MAOA, respectively, to regulate extracellular norepinephrine availability. (a) Representative images of ex vivo SCG explant cultures. Top, the area of the sympathetic ganglia is represented using the reflected-light differential interference contrast (DIC) channel. Bottom, Cx3cr1-GFP+ cells in the same explant culture (GFP channel). Images are representative of 20 similar experiments. Scale bar, 100 μm. (b) Schematic representation of optogenetic activation of sympathetic SCG explant culture (left) followed by CD45.2 (PE)+F4/80 (Alexa Fluor 647)+ cell sorting (right). FSC, forward scatter; SSC, side scatter. (c) NE content in CD45.2+F4/80+ cells isolated from SCG explant cultures from Th-cre; LSL-ChR2-YFP and LSL-ChR2-YFP mice after optogenetic activation. Each data point represents tissues pooled from six mice. n=3-7 experiments. The following numbers of cells were used in NE assays (run in duplicate): 189±30 from Th-cre; LSL-ChR2-YFP SCG (n=7), 126±21 from LSL-ChR2-YFP SCG (n=6), and 159±19 from Th-cre; LSL-ChR2-YFP SCG stimulated with SLC6A2 blocker (n=3). (d) Ex vivo NE release upon optogenetic stimulation of SCG explants isolated from Th-cre; LSL-ChR2-YFP and LSL-ChR2-YFP mice. Each data point represents medium collected from one explant culture. n=7 per group. (e) NE content in CD45.2+F4/80+ cells isolated from the SCG of either B6 or Slc6a2−/− mice and then incubated with ACh, ACh and SLC6A2 blocker, ACh and MAOA blocker, or culture medium. Each data point represents tissues pooled from six mice. n=3-7 experiments. The following numbers of cells were used in NE assays (run in duplicate): 364±128 from B6 SCG (n=7), 238±55 from Slc6a2−/− SCG (n=3), 216±58 from B6 SCG incubated with ACh (n=7), 201±63 from Slc6a2−/− SCG incubated with ACh (n=3), 196±18 from B6 SCG incubated with ACh and SLC6A2 blocker (n=5), and 133±11 from B6 SCG incubated with ACh and MAOA blocker (n=7). (f) Ex vivo NE release from the SCG of either B6 or Slc6a2−/− mice after incubation with ACh, ACh and SLC6A2 blocker, ACh and MAOA blocker, or culture medium. Each data point represents medium collected from one explant culture. n=7 per group. (g) Expression of mRNA as determined by qRT-PCR relative to Gapdh expression for proinflammatory genes (Tnfa and ll1) in CD45.2+F4/80+ cells isolated from SCG explant cultures from Th-cre; LSLChR2-YFP (blue) and LSL-ChR2-YFP (black) mice. Prior to cell sorting, SCG explants were optogenetically stimulated. n=3-4 experiments (for Tnfa, n=4, P=0.0467; for ll1, n=3, P=0.011). (h) Expression of mRNA as determined by qRT-PCR relative to Gapdh expression for anti-inflammatory genes (ll4ra and Arg1) in CD45.2+F4/80+ cells isolated from SCG explant cultures from Th-cre; LSL-ChR2-YFP (blue) and LSL-ChR2-YFP (black) mice. Prior to cell sorting, SCG explants were optogenetically stimulated. n=3-4 experiments (for ll4ra, n=3, P=0.0257; for Arg1, n=4, P=0.0497). Data in c-h were analyzed by two-tailed unpaired Student's t-test and are shown as average±s.e.m. *P<0.05, **P<0.01, ****P<0.0001.

FIG. 2 shows obesity-induced accumulation of SAMs. (a) Representative histograms showing percentages of F4/80 (Alexa Fluor 647)+ cells in sympathetic nerve fibres (left), subcutaneous adipose tissue (middle), and spleen (right) in mice that were genetically obese (ob/ob; black), obese due to HFD (red), ND fed (blue), or fasted for 24 h (green). CD45.2 (PE)+ cells were gated. Histograms are representative of four independent experiments. HFD no Ab, cells without antibody staining harvested from mice fed a HFD. Black lines indicate the region defining F4/80+ cells. (b) Percentages of F4/80 (Alexa Fluor 647)+CD11c (FITC)+ cells in sympathetic nerve fibres (left), subcutaneous adipose tissue (middle), and spleen (right) in mice that were genetically obese (ob/ob; black), obese due to HFD (red), ND fed (blue), or fasted for 24 h (green). CD45.2 (PE)+ cells were gated. n=4 experiments per group. Each data point represents one experiment. (c) Expression of mRNA as determined by qRT-PCR relative to Gapdh expression for proinflammatory genes (Tnfa and ll1) in CD45.2+F4/80+ cells in sympathetic nerve fibres (SAMs), subcutaneous adipose tissue (ATMs), and spleen (SpMs) isolated from mice that were fed either ND (blue) or HFD (red). n=4 experiments per group. Each data point represents tissues pooled from ten mice. (d) Expression of mRNA as determined by qRT-PCR relative to Gapdh expression for anti-inflammatory genes (Arg1 and ll10) in CD45.2+F4/80+ cells including SAMs, ATMs, and SpMs isolated from mice that were fed either ND (blue) or HFD (red). n=4 experiments per group. Each data point represents tissues pooled from ten mice. (e) Heat map showing the expression of pro- and anti-inflammatory genes as determined by the qRT-PCR analyses in c and d. Data in b were analyzed by one-way ANOVA followed by Bonferroni multiple-comparisons test with ND as the control group. Data in c and d were analyzed by two-tailed unpaired Student's t-test. Data are shown as average±s.e.m. **P<0.01, ***P<0.001, ****P<0.0001; ns, not significant.

FIG. 3 shows that the loss of Slc6a2 function in SAMs rescues the thermogenic capacity of ob/ob mice. (a) Schematic representation of bone marrow transplant from either Slc6a2−/− or control B6 (CD45.1) mice into genetically obese ob/ob mice (ob/ob-Slc6a2−/− and ob/obCtrl chimeras, respectively). (b) Rectal temperature of ob/obCtrl (black) and ob/ob-Slc6a2−/− (green) chimeras was measured at room temperature (RT) and after 2 h of cold challenge (4° C.). Each data point represents one mouse. n=4 ob/ob-Slc6a2−/− mice and n=6 ob/obCtrl mice. *P=0.025, ****P<0.0001. (c) Serum levels of NE in ob/obCtrl (black) and ob/ob-Slc6a2−/− (green) chimeras were measured at room temperature and after 2 h of cold exposure (4° C.). Each data point represents one mouse. n=4 mice per group for ob/ob-Slc6a2−/− mice and n=5 mice per group for ob/obCtrl mice. *P=0.022, **P=0.0072. (d) Optical micrographs of BAT removed from ob/ob chimeras following 2 h of cold challenge (4° C.) and stained with H&E. Left, BAT from an ob/obCtrl chimera. Right, BAT from an ob/ob-Slc6a2−/− chimera. Images are representative of fat organs collected from four ob/obCtrl and six ob/ob-Slc6a2−/− mice. (e) Expression of mRNA for Ucp1 as determined by qRT-PCR relative to Gapdh expression in BAT (left) and sWAT (right) dissected after 2 h of cold challenge (4° C.). Each data point represents one mouse. n=4 ob/ob-Slc6a2−/− mice (green) and n=5 ob/obCtrl mice (black). *P=0.0269, **P=0.0015. (f) Optical micrographs of BAT dissected from ob/obCtrl (left) and ob/ob-Slc6a2−/− (right) chimeras following 2 h of cold challenge (4° C.) and stained with anti-UCP1 antibody. Images are representative of fat organs collected from four ob/obCtrl and six ob/ob-Slc6a2−/− mice. (g) Optical micrographs of sWAT dissected from ob/obCtrl (left) and ob/ob-Slc6a2−/− mice (right) following 2 h of cold challenge (4° C.) and stained with anti-UCP1 antibody. Images are representative of fat organs collected from four ob/obCtrl and six ob/ob-Slc6a2−/− mice. (h) Average adipocyte diameter quantified from optical micrographs of sWAT and BAT from ob/ob chimeras following 2 h of cold challenge (4° C.). Measurements are representative of four (ob/ob-Slc6a2−/−) and six (six ob/obCtrl) independent micrographs. 18-34 measurements were obtained per micrograph.n=169 cells for ob/obCtrl sWAT, n=120 cells for ° blob-Slc6a2−/− sWAT, n=180 cells for ob/obCtrl BAT, n=120 cells for ob/ob-Slc6a2−/− BAT. ****P <0.0001. (i) Body weight change (top) and daily food intake (bottom) of ob/obCtrl (n=4 mice) and ob/ob-Slc6a2−/− (n=6 mice) chimeras monitored for 7 weeks following 2 weeks of food intake normalization (0.06 g of food per 1 g of body weight per day; gray shading) that started 9 weeks after bone marrow transplant. The yellow triangle indicates when irradiation was performed. *P<0.05. (j) Blood plasma nonesterified (free) fatty acid (FFA) concentration in ob/obCtrl and ob/ob-Slc6a2−/− chimeras measured 8 weeks after bone marrow transplant before and while mice were under a regimen of 0.06 g of food per 1 g of body weight per day. n=5 mice per group. **P=0.0022. Data in b, c, e, h, and j were analyzed by two-tailed unpaired Student's t-test and in i by multiple t-tests (one Student's t-test per row with correction for multiple comparisons using the Holm-Sidak method). Data are shown as average±s.e.m. Scale bars in d, f, and g, 100 μm.

FIG. 4 shows that SNS is a direct and necessary target of AMPH that mediates its anti-obesity effect, independently of hypophagia and hyperkinesia. (a) sequence of representative pseudocolor images showing calcium levels ([Ca2+]) of one GCaMP3⁺ superior cervical ganglia neuron after stimulation with 10 μM acetylcholine (ACh) for 40 s (arrow). In each frame, the timing after the onset of ACh application is indicated. Changes in fluorescence (ΔF) were measured as relative elevation from baseline fluorescence and expressed as ΔF/F0=[(Fpost−Frest)/Frest] and are represented as pseudocolor scale. (b) representative ACh-induced [Ca2+]i elevation response tracings in Vehicle and AMPH-treated neurons. (c) amplitude of ACh-induced Ca2+ transients in control and after pharmacological treatment with AMPH (***p<0.001; n=8; one-way ANOVA followed by Bonferroni correction). (d) change in Body Weight (ΔBW) of Control (CT) and regionally Sympathectomized (Symp) mice during 6 weeks of High Fat Diet (HFD) exposure plus treatment with Phosphate-Buffered Saline (PBS) or Amphetamine (AMPH) (dose: 0.12 mol/kg of BW, daily IP injections). (e) daily food intake during HFD exposure and respective treatment. (f) representative tracking of the locomotor activity of both Control and Symp mice, measured 1 h post-injection. (g) total distance traveled in 10 min, 1 h post-injection. (*p<0.05; ***p<0.001; ****#### p<0.0001, n=5-10. Statistics done using unpaired Student's t-test, with Holm-Sidak correction method. *PBS vs AMPH; # Control vs Symp). Data presented as mean±S.E.M.

FIG. 5 shows that sympathomimetic action of AMPH is required for its anti-obesity effect and the elevation of lipolysis. FIG. 5A. Representative traces of changes in membrane potential and action potential (AP) evoked under current-clamp mode by injection 500-ms current pulses (−25 to +275 pA in 25 pA increments) from an initial holding potential (Vh) of −70 mV in Vehicle and AMPH treatment. FIG. 5B. Maximum AP firing frequency of Vehicle and AMPH-treated neurons and Resting membrane potential of Vehicle and AMPH-treated neurons (***p<0.001; n=5-8; one-way ANOVA followed by Bonferroni correction). FIG. 5C. Body weight of Control (left) and Symp (right) mice during 6 weeks of HFD exposure and PBS or AMPH treatment (dose: 0.12 mol/kg of BW, daily IP injections). FIG. D. Plasma Triglycerides (TGs), Free Fatty Acids (FFAs) and Glycerol content in HFD fed Control and Symp mice 2 h post-injection without access to food. (*p<0.05; **p<0.01; ***p<0.001; n=5-6. Statistics done using unpaired Student's t-test, with Holm-Sidak correction method. *PBS vs AMPH; ^{#} Control vs Symp). Data presented as mean±S.E.M.

FIG. 6 shows that pegylation of Amphetamine (PEGyAMPH) prevents access to the brain, without compromising its sympathomimetic action. (a) representative scheme of the AMPH's PEGylation method to produce PEGyAMPH. (b) representative mass spectrometry using Fourier-transform ion cyclotron resonance (FT-ICR) of Brain extracts from C57BL/6 mice 30 min post-injection with PBS, AMPH or PEGyAMPH (dose: 0.12 mol/kg of BW for both drugs, IP). Only AMPH replicates showed the expected mass. (c) representative traces of changes in membrane potential and action potential (AP) evoked under current-clamp mode by injection 500-ms current pulses (−25 to +275 pA in 25 pA increments) from an initial holding potential (Vh) of −70 mV in Control, AMPH and PEGyAMPH treatment. (d) maximum AP firing frequency of Control, AMPH and PEGyAMPH-treated neurons. (e) sequence of representative pseudocolor images showing [Ca²⁺]ᵢ changes of one GCaMP3⁺ superior cervical ganglia neuron after stimulation with 10 μM Ach for 40 s (arrow). In each frame, the timing after the onset of ACh application is indicated. Changes in fluorescence (ΔF) were measured as relative elevation from baseline fluorescence and expressed as ΔF/F₀=[(Fₚₒₛₜ−Fᵣₑₛₜ)/Fᵣₑₛₜ] and are represented as pseudocolor scale. (f) representative ACh-induced [Ca²⁺]ᵢ elevation response tracings in control, AMPH and PEGyAMPH-treated neurons. (g) amplitude of ACh-induced Ca²⁺ transients in control and after pharmacological treatment with AMPH and PEGyAMPH. (***p<0.001; n=3-4; one-way ANOVA followed by Bonferroni correction). Data presented as mean±S.E.M.

FIG. 7 shows that PEGyAMPH activates SNS Neurons. (a) resting membrane potential (n=3-4). (b) AP firing threshold and (c) current input for firing of Control, AMPH and PEGyAMPH-treated neurons (*p<0.05; **p<0.001; *** p<0.001; n=4; one-way ANOVA followed by Bonferroni correction). Data presented as mean±S.E.M.

FIG. 8 shows that PEGyAMPH is a peripheral sympathomimetic compound that does not induce hypophagia nor hyperkinesia. (a) Food intake of C57BL/6 mice for 24 h post-injection of PBS, AMPH or PEGyAMPH (dose: 0.12 mol/kg of BW for both drugs, IP). (b) Total distance traveled in 15 min, measured 1 h post-injection. (c) Representative tracking of the locomotor activity of both Control and Symp mice, measured 1 h post-injection with PBS or AMPH. (d) Norepinephrine (NE) content in gonadal and inguinal White Adipose Tissue (gWAT and iWAT, respectively) and (e) Liver of C57/BL6 mice 2 h post-injection with PBS, AMPH or PEGyAMPH without access to food. (*# p<0.05; ****#### p<0.0001, n=4-7. Statistics done using unpaired Student's t-test, with Holm-Sidak correction method. *PBS vs PEGyAMPH; # PBS vs AMPH.) Data presented as mean±S.E.M.

FIG. 9 shows that PEGyAMPH does not affect intestinal absorption of dietary lipids as AMPH does. (A) Plasma triglycerides (TGs) levels of HFD fed C57BL/6 mice 2 h post-injection with PBS, AMPH or PEGyAMPH (dose: 0.12 mol/kg of BW for both drugs, IP) without access to food. (b) Daily Total Faecal output and TGs content. (# p<0.05; ## p<0.001; n=5-8. Statistics done using unpaired Student's t-test, with Holm-Sidak correction method. # PBS vs AMPH.) Data presented as mean±S.E.M.

FIG. 10 shows that PEGyAMPH protects mice from Diet Induced Obesity (DIO), without inducing hypophagia nor hyperkinesia. (A) Change in Body Weight (ΔBW) of C57BL/6 mice during 10 weeks of HFD exposure plus chronic treatment with PBS, AMPH or PEGyAMPH (dose: 0.12 mol/kg of BW for both drugs, daily IP injections). (b) Daily food intake during HFD exposure and respective treatment. (c) Normalised tissue weights after 10 weeks of HFD exposure and respective treatment. (d) Daily Locomotor Activity (LA) during HFD exposure and respective treatment. (e) Cumulative LA for 72 h, measured during the fourth week of HFD exposure and respective treatment. (*, # p<0.05; ### p<0.001; ****, #### p<0.0001, n=5-10. Statistics done using unpaired Student's t-test, with Holm-Sidak correction method. *PBS vs PEGyAMPH; # PBS vs AMPH.) Data presented as mean±S.E.M.

FIG. 11 shows that PEGyAMPH improves peripheral metabolism during DIO. (a) Blood Glucose and (b) Plasma Insulin levels of C57BL/6 mice after 10 weeks of HFD exposure and chronic treatment with PBS, AMPH or PEGyAMPH (dose: 0.12 mol/kg of BW for both drugs, daily IP injections). (c) Levels of Insulin Receptor (IR) and Glucose Transporter type 4 isoform (GLUT4) mRNA expression in the Muscle and Brown Adipose Tissue (BAT) determined by qRT-PCR relative to housekeeping gene Arbp0. (d) and (e). Liver gene expression levels of IR and gluconeogenic genes Glucose 6-phosphatase (G-6-Pase) and Phosphoenolpyruvate carboxykinase (PEPCK) (d), and Lipid metabolism genes Fatty Acid Transporter (FAT), Lipoprotein Lipase (LPL) and Fatty Acid Synthase (FAS) (e) determined by qRT-PCR relative to housekeeping gene GAPDH. (f) Representative Histologic Slices of Livers with Oil-Red (OR)-Staining and (g) Liver TGs content. (*, # p<0.05; **, ## p<0.01; ***, ### p<0.001; ****, #### p<0.0001, n=4-6. Statistics done using unpaired Student's t-test, with Holm-Sidak correction method. *PBS vs PEGyAMPH; # PBS vs AMPH.) Data presented as mean±S.E.M.

FIG. 12 shows that PEGyAMPH elevates Lipolysis during DIO. A. NE content in iWAT, of C57BL/6 mice after 10 weeks of HFD exposure and chronic treatment with PBS, AMPH or PEGyAMPH (dose: 0.12 mol/kg of BW for both drugs, daily IP injections). (b) and (c) Plasma levels of FFAs ((b)) and Glycerol ((c)) of C57BL/6 mice 2 h post-injection with PBS, AMPH or PEGyAMPH without access to food, measured during the fourth and fifth weeks of HFD exposure and respective treatment. (d) Representative Histologic Slices of iWAT stained with haematoxylin and eosin (H&E) and (e) quantification of iWAT Adipocyte Size of C57BL/6 mice after 10 weeks of HFD exposure and chronic treatment with PBS, AMPH or PEGyAMPH. (f) and (g) Lipolytic gene expression levels of beta-3 adrenergic receptor (ADRB3), Adipose triglyceride lipase (AtgL) and Hormone-Sensitive Lipase (HSL) in iWAT (f) and in Brown Adipose Tissue (BAT) (g). determined by qRT-PCR relative to housekeeping gene Arbp0. (*, # p<0.05; **, ## p<0.01; ***, ### p<0.001; ****. #### p<0.0001, n=4-6. Statistics done using unpaired Student's t-test, with Holm-Sidak correction. *PBS vs PEGyAMPH; # PBS vs AMPH.) Data presented as mean±S.E.M.

FIG. 13 shows that PEGyAMPH elevates Lipolysis during DIO. (a) NE content in the Muscle of C57BL/6 mice after 10 weeks of HFD exposure and chronic treatment with PBS, AMPH or PEGyAMPH (dose: 0.12 mol/kg of BW for both drugs, daily IP injections). (b) Muscle mRNA expression levels of lipid metabolism genes determined by qRT-PCR relative to housekeeping gene GAPDH. (*^{#} p<0.05; **^{##} p<0.01; n=4-6. Statistics done using unpaired Student's t-test, with Holm-Sidak correction. *PBS vs PEGyAMPH; # PBS vs AMPH.) Data presented as mean±S.E.M.

FIG. 14 shows that PEGyAMPH elevates Thermogenesis during DIO, without the induction of hyperthermia. (a)-(d) Infrared thermography analysis was performed 2 h post-injection with PBS, AMPH or PEGyAMPH (dose: 0.12 mol/kg of BW for both drugs, IP) on the fourth week after HFD exposure and respective treatment. (a) BAT temperatures. Arrows indicate the region of interest. (b) Quantification of BAT Temperature measured with thermography. (c) Tail temperatures measured 0.5 cm from the tail base. Arrows indicate the region of interest. (d) Quantification of Tail Temperature measured with thermography. (e) BAT mRNA expression levels of thermogenic genes determined by qRT-PCR relative to housekeeping gene Arbp0. after 10 weeks of HFD exposure and chronic treatment with PBS, AMPH or PEGyAMPH. (f) Core Body Temperature was measured with rectal probe 2 h post-injection, on the fourth week after HFD exposure and respective treatment. (*# p<0.05; **, ## p<0.01; ***, ### p<0.001; ****, #### p<0.0001, n=4-8. Statistics done using unpaired Student's t-test, with Holm-Sidak correction. *PBS vs PEGyAMPH; # PBS vs AMPH.) Data presented as mean±S.E.M.

FIG. 15 shows that PEGyAMPH elevates Thermogenesis during DIO. (a) Representative Histologic Slices of H&E-stained BAT and (b) quantification of BAT Adipocyte Size of C57BL/6 mice after 10 weeks of HFD exposure and chronic treatment with PBS, AMPH or PEGyAMPH (dose: 0.12 mol/kg of BW for both drugs, daily IP injections). (c) NE content in BAT. (d) iWAT mRNA expression levels of thermogenic genes determined by qRT-PCR relative to housekeeping gene Arbp0. (*# p<0.05; **## p<0.01; ***### p<0.001; ****#### p<0.0001, n=4-6. Statistics done using unpaired Student's t-test, with Holm-Sidak correction. *PBS vs PEGyAMPH; # PBS vs AMPH.) Data presented as mean±S.E.M.

FIG. 16 shows % increase in the body weight of mice on a high fat diet treated with AMPH, pegAMPH and control.

FIG. 17 shows % change in heart rate of mice treated with AMPH, pegAMPH and control.

### DETAILED DESCRIPTION

This invention relates to the finding that blocking the activity of Solute carrier family 6 member 2 (Slc6a2) outside the brain, and in particular in sympathetic neuron-associated macrophages (SAMs) within adipose tissue, for example using compounds that do not cross the blood brain barrier, exerts a sympathomimetic effect that promotes weight loss and/or inhibits weight gain without adverse cardiac or other CNS mediated effects. Inhibition of Slc6a2 outside the brain is further shown herein to exert a cardio-protective effect.

A compound for use as described herein may comprise a Slc6a2 inhibitor. Slc6a2 (Gene ID: 6530, also referred to as NET; norepinephrine transporter) is a transmembrane protein responsible for reuptake of norepinephrine into presynaptic nerve terminals and is a regulator of norepinephrine homeostasis. Human Slc6a2 may have the reference amino acid sequence of NCBI database entry NP_001034.1 and may be encoded by the reference nucleic acid sequence of NCBI database entry NM_001043.3.

A Slc6a2 inhibitor selectively reduces or inhibits the activity of Slc6a2. Suitable Slc6a2 inhibitors may inhibit the reuptake of norepinephrine into presynaptic terminals.

Suitable Slc6a2 inhibitors for use in the compounds and conjugates described herein are well known in the art and include Amitriptyline, Amoxapine, Amphetamine, a substituted amphetamine, Asenapine maleate, amedalin, Atomoxetine, Bicifadine Hydrochloride, (S,S)-Hydroxy Bupropion, Bupropion HCl, Chlorphenamine, Citalopram, Clomipramine, Cocaine, CP39332, Daledin, Debrisoquin, Desipramine hydrochloride, Desvenlafaxine succinate monohydrate, Dexmethylphenidate, Dextroamphetamine, Dextromethorphan, Diethylpropion, Dopamine, Dosulepin, Doxepin, Droxidopa, Duloxetine, Ephedra, Ephedrine, Ergotamine, Etoperidone, edivoxetine, esreboxetine, GBR 12935 dihydrochloride, Ginkgo biloba, Guanadrel, Guanethidine, Imipramine hydrochloride, Imipramine-d6, Indatraline hydrochloride, lobenguane, lobenguane sulfate I-123, lortalamine, Ketamine, Loxapine, Maprotiline Hydrochloride, Mazindol, Methamphetamine, Methylphenidate, Mianserin, Midomafetamine, Milnacipran hydrochloride, Mirtazapine, MMDA, N,O-Bis(trimethylsilyl)trifluoroacetamide, Nefazodone, Nisoxetine hydrochloride, Norepinephrine, Nortriptyline, Orphenadrine, Paroxetine, Pethidine, Phendimetrazine, Phenmetrazine, Phentermine, Protriptyline, Pseudoephedrine, rac Milnacipran Hydrochloride, Rauwolfia serpentina root, reboxetine, Reboxetine mesylate, Safinamide mesylate, Talopram hydrochloride, Talsupram hydrochloride, Tapentadol, Tandamine, Tomoxetine hydrochloride, Tramadol, Trimipramine, Venlafaxine Hydrochloride, Viloxazine and Zotepine and analogues and derivative thereof.

Substituted amphetamines for use as Slc6a2 inhibitors as described herein may include methamphetamine, ephedrine, cathinone, phentermine, bupropion, methoxyphenamine, selegiline, amfepramone, pyrovalerone and 3,4-methylenedioxymethamphetamine.

The skilled person will be aware of other known Slc6a2 inhibitors which may be used in the present invention.

Preferred Slc6a2 inhibitors include amphetamine.

Compounds for use as described herein may not act via the brain or central nervous system, or may predominantly not act via the brain or central nervous system. Preferred compounds do not cross the blood brain barrier (BBB). For example, the compound may be BBB-impermeant.

In some embodiments, a compound for use as described herein may further comprise a BBB blocking moiety. For example, compounds for use as described herein may include a conjugate comprising a Slc6a2 inhibitor and a BBB blocking moiety.

A BBB blocking moiety is a chemical group that blocks, prevents, substantially reduces, or mitigates against the crossing of the BBB and the delivery of the conjugate comprising the Slc6a2 inhibitor to the brain and CNS.

The BBB blocking moiety ensures that the Slc6a2 is not inhibited in the brain or CNS i.e. the inhibitor does not act via the brain, or predominantly does not act via the brain. BBB blocking moieties may for example increase the size and/or hydrophilicity of the conjugate and/or its localization at fat tissue, thereby blocking, preventing, reducing or mitigating against crossing the blood-brain barrier. In some preferred embodiments, the BBB blocking moiety may increase the hydrodynamic radius and polarity of the conjugate, increasing its hydrophilicity.

BBB blocking moieties may for example include polymer chains, such as (poly)alkylene oxide or a peptide, such as charged peptide chains, for example comprising amino acids with acidic side chains or antibody molecules; or nanomaterials.

The BBB blocking moiety is connected to the Slc6a2 compound using suitable available functionality within the Slc6a2 compound. For example, many of the Slc6a2 compounds for use in the present invention include amino functionality, as this may serve as a site for forming a connection to the BBB blocking moiety. Typically where amino functionality is present this forms a link to a BBB blocking moiety in the form of an amide bond, where the amino group is permitted to react with a carboxylic acid group present within the BBB blocking moiety. Other functionalities may be used, such as carboxylic acid or hydroxyl functionality, as appropriate.

If needed, functionality within the Slc6a2 compound may be modified to allow for the formation of a suitable connection to a BBB blocking moiety. In some embodiments, the connection between the BBB blocking moiety and the Slc6a2 compound may be a triazole group. Such as derived from a click reaction between alkyne and azide coupling partners. To allow for such functionality, the Slc6a2 compound may be modified to include alkyne or azide functionality.

In one embodiment, the BBB blocking moiety may be formed in vivo, although this is less preferred. Here, a conjugate may be provided having the Slc6a2 compound connected to a carrier protein-binding group, such as binding group for albumin or an antibody. When the conjugate is administered, the carrier protein-binding group may bind to a carrier protein-binding group to form a BBB blocking moiety. The carrier protein-bind group is provided with functionality suitable for binding to a carrier protein. In one embodiment the carrier protein-binding group may be provided with functionality suitable for binding with a thiol functionality within a cysteine amino acid residue of the carrier protein-binding group.

By way of example, albumin may be used as a carrier protein and the cysteine residue at position 34 may be used as the binding point between the carrier protein and the carrier protein-binding group of the conjugate.

Such strategies have been described previously, for example by Dumelin et al. (Angew. Chem. Int. Ed. 2008, 47, 3196).

The BBB blocking moiety may be covalently linked to the Slc6a2 inhibitor either directly or through a chemical linker.

In some embodiments, the BBB blocking moiety may be or comprise a polyalkylene oxide. Typically the polyalkylene oxide is polyethylene oxide (also known as polyethylene glycol) or polypropylene oxide. In some preferred embodiments, the BBB blocking moiety may be or comprise a polyethylene glycol (PEG) chain, for example a polyethylene glycol (PEG) chain having 4 or more, 8 or more, 16 or more or 32 or more monomer units. The number of monomer units may be an average number of monomer units.

Where a polyalkylene oxide group is present with the BBB blocking moiety this may be connected to the Slc6a2 inhibitor either directly or through a chemical linker via the terminal functionality of the polyalkylene oxide group, which may be oxygen functionality, or some other functionality.

The polyalkylene oxide group may be connected to the Slc6a2 inhibitor via an amide bond. The polyalkylene oxide group may be provided with a carboxylic group-derived group at a terminal for formation of the amide with amino functionality of the Slc6a2 inhibitor. Here, the preferred Slc6a2 inhibitors for use in the conjugate have amino functionality, and that functionality may be used to connect the Slc6a2 inhibitor to the BBB blocking moiety.

The polyalkylene oxide group may be connected to the Slc6a2 inhibitor via a triazole group. Such a group is typically formed in a click-style reaction in the coupling of an alkyne-containing reagent with an azide-containing partner. Here, one of the polyalkylene oxide group and the Slc6a2 inhibitor may have derived from an alkyne-containing reagent and the other from an azide-containing partner.

A second terminal of the polyalkylene oxide group may have functionality such as hydroxyl, amino or carboxylic acid functionality. This functionality may be used to connect the BBB blocking moiety to other groups. For example, the second terminal of the polyalkylene oxide group may be connected to a targeting moiety, as explained in further detail below. This connection to the other groups may be an amide bond.

Here, the second terminal may be provided with an amine-derived group for formation of the amide with carboxylic acid functionality present within those other groups, for example within the targeting moiety.

In some embodiments, the BBB blocking moiety may be or comprise a peptide group. Here, the peptide group is a plurality of contiguous amino acid residues, which typically include one or more, such as all, amino acid residues having acidic or basic side chains, such as acidic side chains. It is preferred therefore that the peptide groups is a charge group.

The peptide group may have 2 or more, 4 or more, 8 or more, 16 or more or 32 or more amino acid residues.

An amino acid residues typically refers to an α-amino acid residue. This α-amino acid residue may have an acidic side chain, and more specifically a side chain containing or more, such as one or two, carboxylic acid groups.

An amino acid residue may be a natural (proteinogenic) amino acid residue, such as an amino acid residue selected from the group consisting of residues.

An amino acid residue may also be a non-proteinogenic amino acid, for example an aconitic acid residue.

The peptide group may be linear or branched. A branched peptide group is one where a side chain functionality in one or more amino acid residues within the peptide group, such as for those residues having a carboxylic acid group, is connected to another amino acid residue.

In one embodiment, the peptide group contains amino acid residues selected from the group consisting of aspartic acid, glutamic acid and aconitic acid residues.

The peptide group may be connected to the Slc6a2 inhibitor via the carboxy functionality of an amino acid residue, such as the α-carboxy functionality of an amino acid residue. Typically, the peptide group is connected to the Slc6a2 inhibitor via the α-carboxy functionality of a terminal amino acid residue within the peptide group. Thus, the N terminal forms the connection with the Slc6a2 inhibitor.

The peptide group may also be connected to a targeting moiety, and this connection may be formed via amino of carboxyl functionality with the peptide group, and most preferably via amino functionality.

As described in further detail below, the targeting moiety may itself contain one or more amino acid residues, and a peptide group in the BBB blocking moiety these may be connected to the targeting moiety through the amino acid residues in the moiety.

For example, where the targeting moiety is folate, the targeting moiety may connect to the BBB blocking moiety via the glutamic acid residue of the folate, for example via the side chain carboxylic acid functionality of the glutamic acid residue.

In other embodiments, the BBB blocking moiety may comprise both a polyalkylene oxide group and a peptide group, which may be linearly arranged, for example between the Slc6a2 inhibitor and the targeting group, where such is present. Alternatively, one of the polyalkylene oxide group and the peptide group may be provided between the Slc6a2 inhibitor and the targeting group, and the other may be grafted as a side group on the one of the polyalkylene oxide group and the peptide group.

A preferred compound may be a conjugate comprising amphetamine and polyethylene glycol (i.e. PEGylated amphetamine (pegAMPH). The amphetamine is connected to the polyethylene glycol group via the amino functionality of the amphetamine.

In some preferred embodiments, a compound for use as described herein may be targeted to macrophages, most preferably to SAMs which are shown herein to be present in adipose tissue. In other embodiments, a compound for use as described herein may be targeted to adipose tissue. This may improve the safety profile of the compound, particularly in respect to cardiac health.

A compound for use as described herein may further comprise a targeting moiety which facilitates delivery of the compound. For example, a compound for use as described herein may comprise a Slc6a2 inhibitor, a BBB blocking moiety and a targeting moiety.

Suitable targeting moieties include antibody molecules and ligands which bind specifically to surface markers of macrophages, such as folate receptor (FR), F4/80 and Mac1. Preferred targeting moieties may include folate, which specifically binds to FR.

In some preferred embodiments, a compound for use as described herein may comprise a Slc6a2 inhibitor, a BBB blocking moiety and targeting moiety that binds to FR, such as folate. The combination of Slc6a2 and FR provides selectivity for SAMs.

The targeting moiety may be covalently linked to the Slc6a2 inhibitor and/or the BBB blocking moiety either directly or through a chemical linker.

As noted above, where the targeting moiety is folate, this may be connected via the glutamic acid residue, such as via the carboxylic acid group within the side chain of the glutamic acid residue. Where the targeting moiety contains a peptide, such as where the targeting moieties is an antibody molecule, the targeting moiety may be connected via any appropriate free functionality within that moiety, such as the amino and carboxylic acid functionality within the amino acid residues, or via the functionality of the side chains of the amino acid residues. As an example, the targeting moiety may be connected via cysteine residues, using the thiol-functionality of the side chain groups, for instance within a disulfide connection formed with a thiol on the Slc6a2 compound and/or the BBB blocking moiety, or within a thioether connection, for example formed with a maleimide group provided within the Slc6a2 inhibitor and/or the BBB blocking moiety.

In some embodiments, the conjugates of the invention may include cleavable linkers between two or more of the BBB blocking moiety, the Slc6a2 compound, and the targeting moiety. These linkers may be photocleavable, acid or base cleavable, enzyme cleavable, or other. For example the conjugate may contain a protease-cleavable linker, such as valine citruline, which is cleavable by Cathespin B.

Conjugates having cleavable linkers are less preferred, and it is preferred that the conjugates have non-cleavable linkers.

Compounds as described herein may comprise a Slc6a2 inhibitor conjugated to a BBB blocking moiety and optionally a targeting moiety, as described above. Conjugation may be performed by any convenient method, including the use of amide or ester bonds.

Preferred compounds for use as described herein may comprise amphetamine, PEG and folate moieties. Non-limiting examples of compounds comprising amphetamine conjugated to a PEG chain and folate are shown in Table 1.

The molecular weight of the conjugate, which includes the Slc6a2 inhibitor and the BBB blocking moiety, and the targeting moiety, where present, may be at least 1,000, such as at least 1,500, such as at least 2,000, such as at least 2,500. Where appropriate, this molecular weight may be a number average molecular weight, or a weight average molecular weight.

The conjugate may be provided in a protected form. For example, conjugates of the invention includes those having amino acid residues present, for example where the BBB blocking moiety contains a peptide or the targeting moiety includes an amino acid residue. The amino, carboxyl or side chain functionality of the amino acid residues may be protected.

The conjugate may be provided as a solvate, including for example a hydrate.

The conjugate may also be provided as a salt. For example, in the preferred conjugates of the invention an amino acid residue is present within the conjugate, and this may have free amino or carboxylic acid functionality. The conjugate may be provided with the acid and base conjugate salts, which utilise the amino and acid functionality present.

The skilled person will understand that the invention covers compounds which have the functions indicated, and which are not limited to the chemical structures exemplified herein. By “compounds” herein is meant not only small molecules but also larger molecules, for example antibody drug conjugates. Antibodies, for example antibodies specific for macrophages, or directed against surface features of macrophages, may be used as targeting moieties in accordance with the invention.

While it is possible for a compound or conjugate comprising a Slc6a2 inhibitor as described herein to be administered to the individual alone, it is preferable to present the compound in a pharmaceutical composition or formulation.

A pharmaceutical composition may comprise, in addition to the compound comprising a Slc6a2 inhibitor as described herein, one or more pharmaceutically acceptable carriers, adjuvants, excipients, diluents, fillers, buffers, stabilisers, preservatives, lubricants, or other materials well-known to those skilled in the art. Such materials should be non-toxic and should not interfere with the efficacy of the active compound. The precise nature of the carrier or other material will depend on the route of administration, which may be by bolus, infusion, injection or any other suitable route, as discussed below. Suitable materials will be sterile and pyrogen free, with a suitable isotonicity and stability. Examples include sterile saline (e.g. 0.9% NaCl), water, dextrose, glycerol, ethanol or the like or combinations thereof. The composition may further contain auxiliary substances such as wetting agents, emulsifying agents, pH buffering agents or the like.

Suitable carriers, excipients, etc. can be found in standard pharmaceutical texts, for example, Remington's Pharmaceutical Sciences, 18th edition, Mack Publishing Company, Easton, Pa., 1990.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The formulations may conveniently be presented in unit dosage form and may be prepared by any methods well-known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

A compound comprising a Slc6a2 inhibitor as described herein or pharmaceutical compositions comprising the compound may be administered to a subject by any convenient route of administration, whether systemically/peripherally or at the site of desired action, including but not limited to, oral (e.g. by ingestion); and parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Usually administration will be by the oral route, although other routes such as intraperitoneal, subcutaneous, transdermal, intravenous, nasal, intramuscular or other convenient routes are not excluded.

The pharmaceutical compositions comprising a compound described herein may be formulated in a dosage unit formulation that is appropriate for the intended route of administration.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, ascorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example, from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to macrophages or adipose tissue.

Optionally, other therapeutic or prophylactic agents may be included in the pharmaceutical composition or formulation

Compounds comprising a Slc6a2 inhibitor as described herein may be useful in promoting weight loss and/or inhibiting weight gain. This may have a non-therapeutic (e.g. cosmetic or well-being related) or a therapeutic purpose. For example, a compound described herein may be useful in treating obesity or an obesity-related condition in an individual in need thereof.

Obesity is a condition characterised by the excess accumulation of body fat in an individual. Obesity may have a negative impact on the health or well-being of the individual and obese individuals may be at increased risk of morbidity. For example, an obese individual may be at an increased risk of an obesity-related condition compared to non-obese individuals.

Obesity may include Diet Induced Obesity (DIO).

Obesity-related conditions may include cardiac conditions, such as high blood pressure, deep vein thrombosis and coronary heart disease; endocrinal conditions, such as diabetes and polycystic ovarian syndrome; neurological conditions, such as stroke and dementia, rheumatological conditions, such as gout; osteoarthritis; dermatological conditions, such as cellulitis; gastroenterological conditions, such as fatty liver disease; cancer, such as oesophageal, colorectal, pancreatic, or gall bladder cancer or respiratory conditions, such as asthma and obstructive sleep apnea.

Obesity and obesity-related conditions may be identified in an individual using standard diagnostic criteria. For example, an individual identified as having a body mass index (BMI) of greater than 30 kg/m² may be identified as obese. Examples of such clinical standards can be found in textbooks of medicine such as Harrison's Principles of Internal Medicine, 15th Ed., Fauci A S et al., eds., McGraw-Hill, New York, 2001

The patient may have been previously identified as having obesity and/or an obesity-related condition or be at risk of developing obesity and/or an obesity-related condition. In other embodiments, a method may comprise identifying the patient as having or being at risk of developing obesity and/or an obesity-related condition before administration.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

In some preferred embodiments, the individual is a human. In other preferred embodiments, non-human mammals, especially mammals that are conventionally used as models for demonstrating therapeutic efficacy in humans (e.g. murine, primate, porcine, canine, or leporid) may be employed.

Treatment may be any treatment or therapy, whether of a human or an animal (e.g. in veterinary applications), in which some desired therapeutic effect is achieved, for example, the inhibition or delay of the onset or progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or individual beyond that expected in the absence of treatment. For example, an individual treated as described herein may display reduced or stable weight, reduced body fat and/or a reduced body mass index.

Treatment as described herein may include prophylactic treatment (i.e. prophylaxis) i.e. the individual being treated may not have or may not be diagnosed as having obesity and/or an obesity-related condition at the time of treatment. For example, an individual susceptible to or at risk of the occurrence or re-occurrence of obesity and/or an obesity-related condition may be treated as described herein. Such treatment may prevent or delay the occurrence or re-occurrence of the obesity and/or an obesity-related condition in the individual or reduce its symptoms or severity after occurrence or re-occurrence. In some embodiments, the individual may have been previously identified as having increased susceptibility or risk of obesity and/or an obesity-related condition compared to the general population or a method may comprise identifying an individual who has increased susceptibility or risk of obesity and/or an obesity-related condition. Prophylactic or preventative treatment may be preferred in some embodiments.

A compound comprising a Slc6a2 inhibitor as described herein may be administered as described herein in a therapeutically-effective amount. The term “therapeutically-effective amount” as used herein, pertains to that amount of an active compound, or a combination, material, composition or dosage form comprising an active compound, which is effective for producing some desired therapeutic effect, commensurate with a reasonable benefit/risk ratio.

The appropriate dosage of a compound comprising a Slc6a2 inhibitor as described herein may vary from individual to individual. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the active compound, other drugs, compounds, and/or materials used in combination, and the age, sex, weight, condition, general health, and prior medical history of the individual. The amount of active compounds and route of administration will ultimately be at the discretion of the physician, although generally the dosage will be to achieve therapeutic plasma concentrations of the active compound without causing substantial harmful or deleterious side-effects.

In general, a suitable dose of the active compound is in the range of about 100 μg to about 400 mg per kilogram body weight of the subject per day, preferably 200 μg to about 200 mg per kilogram body weight of the subject per day. Where the active compound is a salt, an ester, prodrug, or the like, the amount administered is calculated on the basis of the parent compound and so the actual weight to be used is increased proportionately. For example, 50 to 100 mg of compound comprising a Slc6a2 inhibitor as described herein may be orally administered twice daily in capsule or tablet form.

Administration in vivo can be effected in one dose, continuously or intermittently (e.g., in divided doses at appropriate intervals).

Methods of determining the most effective means and dosage of administration are well known in the art and will vary with the formulation used for therapy, the purpose of the therapy, the target cell being treated, and the subject being treated. Single or multiple administrations can be carried out with the dose level and pattern being selected by the physician.

Multiple doses of the compound comprising a Slc6a2 inhibitor as described herein may be administered, for example 2, 3, 4, 5 or more than 5 doses may be administered. The administration of the compound comprising a Slc6a2 inhibitor as described herein may continue for sustained periods of time. For example treatment with the compound comprising a Slc6a2 inhibitor as described herein may be continued for at least 1 week, at least 2 weeks, at least 3 weeks, at least 1 month or at least 2 months. Treatment with the compound comprising a Slc6a2 inhibitor as described herein may be continued for as long as is necessary to cause weight loss or reduce or eliminate obesity.

The compound comprising a Slc6a2 inhibitor as described herein may be administered alone or in combination with other treatments, either simultaneously or sequentially dependent upon the individual circumstances. For example, a compound comprising a Slc6a2 inhibitor as described herein as described herein may be administered in combination with one or more additional active compounds.

The compound comprising a Slc6a2 inhibitor as described herein may be administered in combination with a second therapeutic agent, such as orlistat, lorcaserin, phentermine, topiramate, buproprion, naltrexone, or liraglutide; a dietary regime, or a surgical intervention, such as bariatric surgery.

It will be understood that the present invention provides compounds for the treatment of obesity and corresponding methods of treatment, but also first medical uses of compounds, and novel compounds per se.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification, as well as the contents of the priority application PT20171000065945, are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### EXPERIMENTAL

The cellular mechanism(s) linking macrophages to norepinephrine (NE)-mediated regulation of thermogenesis has been a topic of debate. Here, we identify sympathetic neuron-associated macrophages (SAMs) as a population of cells that mediate clearance of NE via expression of Slc6a2, an NE transporter, and monoamine oxidase A (MAOa), a degradation enzyme. Optogenetic activation of the SNS upregulates NE uptake by SAMs and shifts the SAM profile to a more pro-inflammatory state. NE uptake by SAMs is prevented by genetic deletion of Slc6a2 or inhibition of the transporter. We also observed increased SAM content in the SNS of two obesity mouse models. Genetic ablation of Slc6a2 in SAMs increases brown adipose tissue (BAT) content, causes browning of white fat, increases thermogenesis, and leads to significant and sustained weight loss of obese mice. We further show that this pathway is conserved, as human sympathetic ganglia also contain SAMs expressing the analogous molecular machinery for NE clearance, thus constituting a potential target for obesity treatment.

### Materials and Methods

### Immunofluorescence and Confocal Microscopy

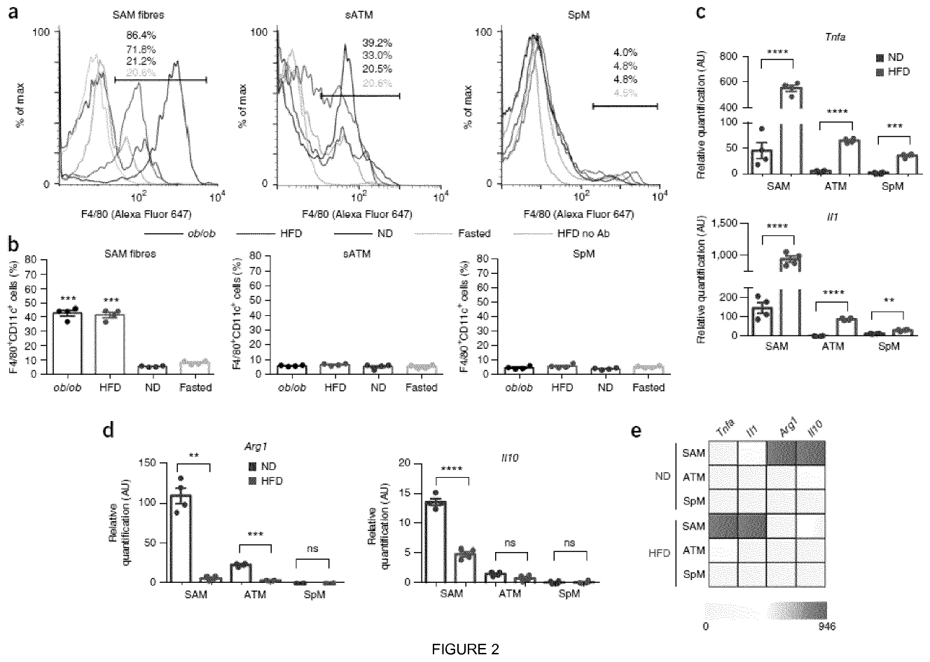

Tissues were dissected and fixed in 4% Paraformaldehyde for 2 hours (at room temperature (RT), with agitation). For images in FIG. 2 j and k we employed frozen sections and the fixation step was followed by cryoprotection in 30% sucrose (Alfa Aesar). 16 μm sections were obtained in a Leica Cryostat CM3050S. Both frozen sections and the whole mount tissues were incubated in a blocking/permeabilization solution (3% Bovine serum albumin, 2% Goat serum, 0.1% Tween and 0.1% Sodium azide in 1×PBS) for 1 hour at RT, with (whole mouns) or without (frozen sections) agitation. Incubations with primary antibodies were performed overnight at 4° C. with (whole mount) or without (frozen sections) agitation. The following dilutions of primary antibodies were used: anti-GFP (1:500), anti-TH (1:1000), anti-Slc6a2 (1:500), anti-MAOa (1:100). Incubation with secondary antibodies was performed for 1-2 hours at RT, with or without (in case of frozen sections) agitation. Z series stacks were acquired on a Leica TCS SP5 confocal Inverted microscope. Analysis and quantification of images were performed in FIJI.

### In Vivo 2-Photon Microscopy

Mice 2 months old were kept anesthetized with 2% isofluorane. During surgery, body temperature was maintained at 37° C. with a warming pad. After application of local anaesthetic (lidocaine), a sagittal incision of the skin was made above the suprapelvic flank to expose the subcutaneous inguinal fat pad. An imaging chamber was custom built to minimize fat movement. Warm imaging solution (in mM: 130 NaCl, 3 KCl, 2.5 CaCl2), 0.6 6H2O, MgCl2, 10 HEPES without Na, 1.2 NaHCO₃, glucose, pH 7.45 with NaOH) (37° C.) mixed with a fat dye (LipidTOX) was applied to label adipocytes, maintain tissue integrity, and to allow the use of immersion objective. Imaging experiments were performed under a two-photon laser-scanning microscope (Ultima, Prairie Instruments Inc.). Live images were acquired at 8-12 frames per second, at depths below the surface ranging from 100 to 250 mm, using an Olympus 20×1.0 N.A. water immersion objective, with a laser tuned to 810-940 nm wavelength, and emission filters 525/50 nm and 595/50 nm for green and red fluorescence, respectively. Laser power was adjusted to be 20-25 mW at the focal plane (maximally 35 mW), depending on the imaging depth and level of expression of GFP and LipidTOX spread. Analysis and quantification of images were performed in FIJI.

### Electron Microscopy.

Fresh tissue was perfused with 2% paraformaldehyde (Electron Microscopy Services (EMS)), 0.2% glutaraldehyde (EMS) in 0.1M phosphate buffer (PB) (pH 7.4). After perfusion, fibres were isolated and immersion fixed for 2 hours at room temperature (RT) in the same fixative. For quenching free-aldehydes auto-fluorescence, nerves were washed with 0.15% glycine (VWR), in PB for 10 minutes at RT.

### Correlative Light-Electron Microscopy (CLEM).

After fixation, the fibres were stabilized with 0.1% tannic acid (EMS) and embed in 2% agarose (Omnipur) before cryoprotection in 30% sucrose (Alfa Aesar) ON at 4° C. Embed samples were placed in optimal cutting temperature (OCT) compound (Sakura) and plunge freeze in liquid nitrogen. 10 μm sections were obtained in a Leica Cryostat CM3050S and placed in cover-glasses coated with 2% (3-Aminopropyl)triethoxysilane (Sigma Aldrich) in acetone. The light microscopy imaging was performed in a Leica SP5 Live microscope after mounting the sections with PB. For electron microscopy processing, samples were washed 10 times with PB and post-fixed in 1% osmium tetroxide (EMS) with 1% potassium hexacyanoferrate (Sigma Aldrich) in PB for 30 minutes, on ice. Dehydration was done in a graded ethanol series of 30%, 50%, 75%, 90% and 100%, for 10 minutes each. EPON resin (EMS) was used for embedding. 70 nm serial sections were obtained in a Leica UC7 and stained with 1% uranyl acetate and lead citrate for 5 minutes each. Electron microscopy images were acquired on a Hitachi H-7650 operating at 100 kV.

### Single Cell Suspension

Tissues were dissected from 10 mice. Spleen, brain, visceral fat and subcutaneous fat were excised and digested for 30 minutes with collagenase (Sigma) at 37° C. with shaking. Sympathetic nerve fibres were isolated from subcutaneous adipose tissues and digested for 30 minutes with Hyaluronidase (Sigma) at 37° C. with shaking, washed and further digested with collagenase for 15 minutes. SCG were dissected and digested with collagenase for 10 minutes, washed and further digested with trypsin (Biowest) for 30 minutes at 37° C. with shaking. Cell suspensions were filtered through a 70 μm sieve and centrifuged at 450×g for 5 minutes.

Flow cytometry.

Flow cytometry data were acquired on a LSR Fortessa X-20 SORP (Becton-Dickinson), FACScalibur (Becton-Dickinson) or Cyan-ADP (Beckman Coulter) and analyzed using FlowJo software package (Tree Star). Macrophages were sorted as live CD45, F4/80-double positive using a FACS Aria llu High Speed cell sorter (Becton Dickinson) or MoFlo High-Speed Cell Sorter produced by Dako Cytomation (now owned by Beckman Coulter).

### Bone Marrow Chimeras.

B6-CD45.1 mice (8-10 weeks), B6 (C57BL/6J) mice (8-10 weeks) or ob/ob (8-10 weeks) mice were lethally irradiated (900 rad, 3.42 minutes, 137Cs source) (Gammacell 2000) and reconstituted with bone marrow cells from either Cx3cr1GFP/+ mice (6 weeks), Slc6a2−/− mice (6-8 weeks), 86 mice (6-8 weeks) or 86-CD45.1 mice (6-8 weeks). B6-CD45.1 mice and B6 mice were reconstituted with 5×10⁶ total bone marrow cells and ob/ob mice were reconstituted with 3×10⁷ total bone marrow cells. Chimerism was assessed 8 weeks after by flow cytometry.

### Low-Input RNAseq Library Preparation.

Sequencing libraries were prepared according to the Smart-seq2 method⁴⁶ with some modifications. 1715±115 cells from nerve fibres, 1534±85 cells from superior cervical ganglia and 5000 cells from other tissues (visceral fat, subcutaneous fat, spleen and brain) were isolated as live CD45+F4/80+ in Trizol (Thermo Fisher) and were used as starting material. RNA was extracted with the Direct-zol MicroPrep kit (Zymo Research) with on-column DNAsel treatment. 10 μL of purified RNA was mixed with 5.5 μL of SMARTScribe 5× First-Strand Buffer (Clontech), 1 μL polyT-RT primer (2.5 μM), 0.5 μL SUPERase-IN (Ambion), 4 μL dNTP mix (10 mM, Invitrogen), 0.5 μL DTT (20 mM, Clontech) and 2 μL Betaine solution (5 M, Sigma), incubated 50° C. 3 min. 3.9 μL of first strand mix, containing 0.2 μL 1% Tween-20, 0.32 μL MgCl2 (500 mM), 0.88 μL Betaine solution (5 M, Sigma), 0.5 μL SUPERase-IN (Ambion) and 2 μL SMARTScribe Reverse Transcriptase (100 U/μL, Clontech) was added and incubated one cycle 25° C. 3 min., 42° C. 60 min. 1.62 μL template switch (TS) reaction mix containing 0.8 μL biotin-TS oligo (10 μM), 0.5 μL SMARTScribe Reverse Transcriptase (100 U/μL Clontech) and 0.32 μL SMARTScribe 5× First-Strand Buffer (Clontech) was added, then incubated at 50° C. 2 min., 42° C. 80 min., 70° C. 10 min. 14.8 μL second strand synthesis, pre-amplification mix containing 1 μL pre-amp oligo (10 μM), 8.8 μL KAPA HiFi Fidelity Buffer (5×, KAPA Biosystems), 3.5 μL dNTP mix (10 mM, Invitrogen) and 1.5 μL KAPA HiFi HotStart DNA Polymerase (1 U/μL, KAPA Biosystems), was added, then amplified by PCR: 95° C. 3 min., 8 cycles 98° C. 20 seconds, 67° C. 15 sec and 72° C. 6 min, final extension 72° C. 5 min. The synthesized dsDNA was purified using Sera-Mag Speedbeads (Thermo Fisher Scientific) with final 8.4% PEG8000, 1.1M NaCl, then eluted with 13 μL UltraPure water (Invitrogen). The product was quantified by Qubit dsDNA High Sensitivity Assay Kit (Invitrogen) and libraries were prepared using the Nextera DNA Sample Preparation kit (Illumina). Tagmentation mix containing 11 μL 2×Tagment DNA Buffer and 1 μL Tagment DNA Enzyme was added to 10 μL purified DNA, then incubated at 55° C. 15 min. 6 μL Nextera Resuspension Buffer (Illumina) was added and incubated at room temperature for 5 min. Tagmented DNA was purified using Sera-Mag Speedbeads (Thermo Fisher Scientific) with final 7.8% PEG8000, 0.98M NaCl, then eluted with 25 μL UltraPure water (Invitrogen). Final enrichment amplification was performed with Nextera primers, adding 1 μL Index 1 primers (100 μM, N7xx), 1 μL Index 2 primers (100 μM, N5xx) and 27 μL NEBNext High-Fidelity 2×PCR Master Mix (New England BioLabs), then amplified by PCR: 72° C. 5 min., 98° C. 30 sec., 8-13 cycles 98° C. 10 seconds, 63° C. 30 sec., and 72° C. 1 min. Libraries were size selected, quantified Qubit dsDNA HS Assay Kit (Thermo Fisher Scientific), pooled and sequenced on a NextSeq 500 (Illumina) for 76 cycles at a depth of 25 to 30 million single end reads per sample. To normalize for genomic DNA contamination, which occurred in some samples due to incomplete DNA removal during RNA isolation, the average intronic noise per base pair in all intronic regions per gene was calculated. The exonic reads were then normalized by subtracting the background noise per base pair for the complete length of the exonic regions. Genes without introns were not normalized, as these genes are the minority of genes and are typically short. Fastq files from sequencing experiments were mapped to the mouse mm10 genome using default parameters for STAR⁴⁷. Mapped data were analyzed with HOMER48, custom R, and Perl scripts.

### Superior Cervical Ganglia (SCG) Explant Cultures.

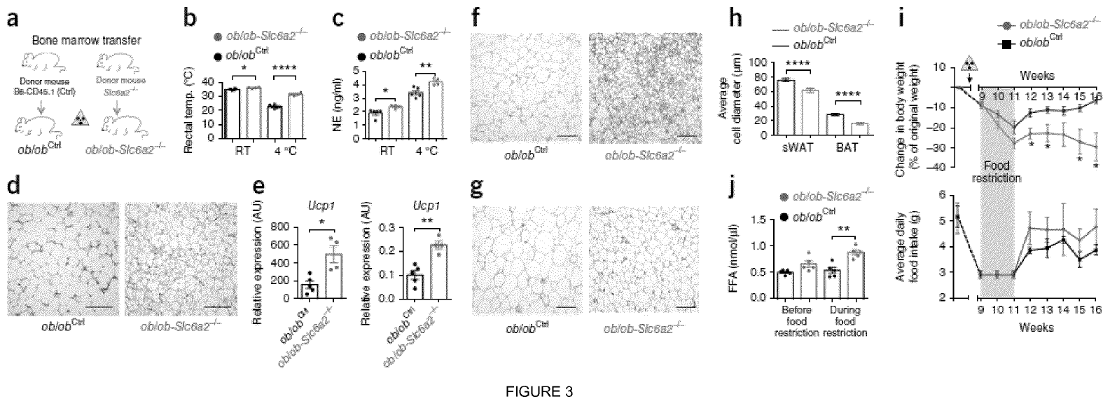

SCG were removed from 4-6 weeks old mice under a stereomicroscope and placed in Dulbecco's Modified Eagle's medium (DMEM, Invitrogen, Carlsbad, Calif., U.S.A.). Ganglia were cleaned from the surrounding tissue capsule and transferred into 8-well Tissue Culture Chambers (Sarstedt, Nümbrecht, Germany) that were previously coated with poly-D-lysine (Sigma/Aldrich, Steinheim, Germany) in accordance to the manufacturers instructions. Ganglia were then covered with 5 μl of Matrigel (BD Bioscience, San Jose, Calif., U.S.A.) and incubated for 7 min at 37° C. DMEM without phenol red (Invitrogen) supplemented with 10% fetal bovine serum (Invitrogen), 2 mM L-Glutamine (Biowest, Nuaillé, France) and nerve growth factor (Sigma/Aldrich) were subsequently added. 12 SCG explants cultures were prepared per condition. SCG ganglia were cultured for minimum 24 hours prior to further manipulation. Stimulation protocol in FIG. 3 was performed for 2 hours with the following concentrations of drugs: 10 mM Acetylcholine chloride, 100 nM Nisoxetine hydrochloride, and 100 μM Clorgyline.

NE Measurements after Optogenetic Stimulation Ex Vivo.

Depolarization of sympathetic neurons in TH-Cre/LSLChR2-YFP explant cultures were performed on a Yokogawa CSUX Spinning Disk confocal using the 488 nm laser line and pointing at the region of interest (ROI) for 200 μs. Stimulation was repeated 7 times using 40% of laser intensity. NE in the SCG explant culture medium and sorted CD45, F4/80-double positive cells was determined with NE ELISA kit (Labor Diagnostika Nord GmbH, Nordhorn, Germany, cat # BA E-5200). The same procedure was performed for LSLChR2-YFP control mice.

NE Measurements in Macrophages from sWAT.

CD45.2-PE, F4/80-Alexa Fluor 647—double positive cells from sWAT were sorted as live and incubated with 2 μM Norepinephrine for 2 hours using the same culture conditions as for SCG explant cultures. Afterwards cells were washed twice with 1×PBS and NE content was measured with NE ELISA kit (Labor Diagnostika Nord GmbH, Nordhorn, Germany, cat # BA E-5200).

### Quantitative PCR.

Total RNA from sorted cells was isolated using RNeasy Plus Micro Kit (Qiagen, cat #50974034). Total RNA from adipose tissues was isolated with PureLink RNA Mini Kit (Ambion, Life Technologies, cat #12183025). cDNA was reverse transcribed using SuperScript II (Invitrogen) and random primers (Invitrogen). Quantitative PCR was performed using SYBR Green (Applied Biosystems) in ABI QuantStudio (Applied Biosystems). GAPDH housekeeping gene was used to normalize samples.

### Functional Studies.

We measured body rectal temperature with an electronic thermometer (Precision) when the animals were housed both at RT and at 4° C. with ND food and water ad libitum. Free fatty acids were measured in blood plasma using Free Fatty Acid Quantitation Kit (Sigma-Aldrich, cat # MAK044-1KT). Serum NE levels were determined with NE ELISA kit (Labor Diagnostika Nord GmbH, Nordhorn, Germany, cat # BA E-5200).

### High-Fat Diet Challenge

When 86 mice reached 8 weeks we replaced ND with HFD (Ssniff, Spezialdiäten GmbH, Soest, Germany), which contains 60 kJ % fat. Analyses were performed when mice gained 40% increase in body weight, after 3 months of HFD.

Intracellular Stain with Ki67.

Cells were surface stained for 30 min. Subsequently, cells were washed and fixed with fixation/permeabilization buffer (eBiosciences) and then permeabilized with permeabilization buffer (eBiosciences). Following this process cells were intracellularly stained with anti-Ki67 or isotype control.

### Histopathological and Immunohistochemical Analysis

The human and mouse tissues were fixed in buffered formalin and the inclusion in paraffin was done according to the standard technical procedures. Histochemical and immunohistochemical studies were performed on formalin fixed paraffin-embedded tissue sections. Sections were 2 microns (human ganglia) or 3-6 microns (mouse tissues) thick (for H&E) and 4 microns thick (for the immunohistochemical study). The following markers were used for immunohistochemistry-aminoethylcarbazole (AEC) and 3, 3′-diaminobenzidine (DAB), accordingly to the usual technical procedure for the marker. For the immunohistochemical studies sections underwent antigenic recovery prior to incubation with primary antibodies—anti-CD68 (Dako; clone PG-M1; dilution 1/150) anti-human Slc6a2 (Mab Techonolgies, clone 3-6C1 sc H10; dilution 1/1000), anti-MAOa (Abcam, clone GR155892-5, dilution 1/50), anti-UCP1 (Abcam, dilution 1/500). Human tissues were analyzed under an optical microscope (Nikon Eclipse 50i) and iconography microscopic images captured using a coupled digital camera (DS Camera Control Unit DS-L2). Mouse tissues were analyzed using Leica DM LB2 microscope and images were captured with Leica DFC 250 camera.

### DT-Mediated Macrophages Depletion

We used LysM-Cre/LSLCSF1R-DTR mice for this experiment and LSL-CSF1R-DTR as controls. Animals received injections of Diphtheria Toxin (DT) from Corynebacterium diphtheria (Calbiochem) once daily for 4 consecutive days. First dose was 500 ng of DT in PBS/20 g of body weight followed by three doses of 250 ng of DT in PBS/20 g of body weight. Depletion was assessed by flow cytometry 12 hours after the fourth injection. NE levels in adipose tissues were assayed with NE ELISA kit (Labor Diagnostika Nord GmbH, Nordhorn, Germany, cat # BA E-5200). Protein concentration was determined by the Bradford Method.

### Mice and Housing Conditions.

Mice (male) 8-18 weeks old were housed at controlled temperature and humidity, under a 12 h light/dark cycle. Food and water were supplied ad libitum, unless mentioned otherwise. The animal experiments were performed in agreement with the International Law on Animal Experimentation and were approved by the IGC ethics committee and by the USC Ethical Committee (Project ID 15010/14/006). C57BL/6 mice were obtained from the Mice Production Facility at the IGC. TH-cre (Jax, #008601), CAG-LSL-GCaMP3 (Jax, #014538), LSL-DTR (Jax, #007900), mice were purchased from Jackson Laboratory, and bred to produce homozygous TH-cre; CAG-LSL-GCaMP3 and TH-cre; LSL-DTR mice. LSL-DTR mice were used as controls for the sympathectomization studies.

### PEGyDT-Mediated Regional Sympathectomy

For detailed characterization refer to Pereira et al. 2017(52). Briefly, TH-cre; LSL-DTR mice were used for this experiment and LSL-DTR mice were used as controls. PEGylated Diphtheria Toxin (PEGyDT) was administered once a day for 8 consecutive days (25 ng/g of BW, IP injections). All following experiments were performed at least 24 h post the last injection.

### PEGylation of Amphetamine (PEGyAMPH Synthesis).

Briefly, in a round-bottom flask, (R)-1-phenylprop-2-ylamine hydrochloride salt (103 mg, 0.6 mmol, 2 eq, Asiba Pharmatec.) was placed under inert atmosphere. A 1.1 mL solution of methyl-PEG-NHS-ester reagent (100 mg, 0.39 mmol, 1 eq, Thermo Scientific) in DMSO was then added, followed by the addition of diisopropylethylamine (DIPEA, 105 μL, 0.6 mmol, 2 eq, Sigma-Aldrich). The reaction was stirred at room temperature for 46 h, after which a multiple extraction with water/ethyl acetate was performed to remove the product from DMSO. Then, a preparative chromatography (EtOAc: MeOH 5%) was performed in order to isolate compound PEGyAMPH in 98% yield (0.1 g). Characterization: ¹H NMR (300 MHz, CDCl₃) δ 7.25-7.11 (m, 5H), 6.53-6.26 (m, 1H), 4.19 (p, J=6.8 Hz, 1H), 3.63-3.47 (m, 14H), 3.32 (s, 3H), 2.79 (dd, J=13.5, 6.1 Hz, 1H), 2.65 (dd, J=13.5, 7.1 Hz, 1H), 2.37 (t, J=6.4 Hz, 2H), 1.06 (d, J=6.6 Hz, 3H). ¹³C NMR (75 MHz, CDCl₃) δ 170.92, 138.38, 129.55, 128.36, 126.40, 72.01, 70.70, 70.60, 70.46, 70.34, 67.43, 59.11, 46.02, 42.60, 37.21. HRMS: [M+H]⁺_{calc}=354.22750; [M+H]⁺ᵣₑₐₗ=354.22783 (error −0.9 ppm). The upscale of the reaction for chronic in vivo treatments was reproduced by Wuxi AppTec.

### SCG Neurons Culture and Treatments.

Primary cultures of SCG neurons were performed from postnatal day 30 C57BL/6 or GCaMP3⁺ mice. After decapitation, both SCG of each animal were removed and cleaned of all visible adipose tissue and surrounding connective tissue before transfer to Dulbecco's Modified Eagle Medium (Biowest). Then, SCG were treated enzymatically in two steps to yield single neurons in accordance to the method described by Motagally and collaborators (32), with some modifications. First, SCG were subjected to enzymatic dissociation in 2.5 mg/mL collagenase solution (Sigma-Aldrich) in Hank's Balanced Salt Solution (HBSS) without calcium and magnesium (Gibco, Life Technologies) at 37° C. with agitation, followed by 0.25% trypsin solution (Biowest) in PBS at 37° C. with agitation. SCG were next mechanically dissociated into a suspension of single cells. The isolated sympathetic neurons were plated, 2500 cells per coverslip (6 mm) coated with poly-d-lysine (Sigma) and growth factor-reduced Matrigel (BD Biosciences) and cultured in Neurobasal medium (Gibco) supplemented with 2% B-27 (Gibco), 10% fetal bovine serum (Gibco), 1% penicillin/streptomycin (Biowest), 100 ng/mL nerve growth factor (AbD Serotec) and 5 μM 5-fluoro-2′-deoxyuridine (Sigma-Aldrich). Cells were kept in culture for 6 days in vitro (DIV) at 37° C. with 5% CO₂ conditioned atmosphere to obtain an enriched culture of sympathetic neurons.

### Intracellular Calcium Imaging.

For Ca²⁺ experiments, sympathetic neurons obtained from GCaMP3⁺ mice. Neurons were incubated with 15 μM AMPH or 15 μM PEGyAMPH for 24 h at 37° C. with 5% CO₂ conditioned atmosphere. At 7 DIV, coverslips with sympathetic neurons from GCaMP3⁺ mice were mounted on an inverted microscope with epifluorescent optics (Axiovert 135TV, Zeiss) equipped with a xenon lamp (located at a Lambda DG-4 (Sutter Instrument) and band-pass filter of 450-490 nm wavelengths. Ca²⁺ measurements were performed at 37° C., as reported in Jacob et al., 2014(33) Throughout the experiments the Ach was applied focally through a drug filled micropipette placed under visual guidance over a single neuronal cell. Drug release was performed by focal pressure (10 psi for 40 s) through a Toohey Spritzer pressure System Ile (Toohey Company). Pressure application of external physiological solution did not cause any measurable change in intracellular Ca²⁺ concentration. Images were obtained every 250 ms by exciting the preparations at 450-490 nm and the emission wavelength was set to 510 nm. Neurons were imaged with a cooled CCD camera (Photometrics CoolSNAP fx), processed and analysed using the software MetaFluor (Universal laging, West Chester, Pa.). Ca²⁺ levels were recorded at the cell body of neurons (manually defined over the cell profile) in the field of view and variations were estimated as changes of the fluorescence signal over the baseline (ΔF/F0=[(Fₚₒₛₜ−Fᵣₑₛₜ)/Fᵣₑₛₜ]).

### Electrophysiology

Whole cell patch-clamp recordings were obtained from 7 DIV dissociated cultures of C57BL/6 mice using an upright microscope (Zeiss Axioskop 2FS) equipped with differential interference contrast optics using a Zeiss AxioCam MRm camera and an ×40 IR-Achroplan objective. During recordings, cells were continuously superfused with artificial cerebrospinal fluid containing (in mM: 124 NaCl, 3 KCl, 1.2 NaH₂PO₄, 25 NaHCO₃, 2 CaCl₂), 1 MgSO₄ and 10 glucose), which was continuously gassed with 95% O₂/5% CO₂. Recordings were performed at room temperature in current-clamp or voltage-clamp mode [holding potential (Vh)=−60 mV] with an Axopatch 200B amplifier (Axon Instruments)(34). Briefly, patch pipettes with 4 to 7 MΩ resistance when filled with an internal solution (containing (in mM): 125 K-gluconate, 11 KCl, 0.1 CaCl₂), 2 MgCl2, 1 EGTA, 10 HEPES, 2 MgATP, 0.3 NaGTP, and 10 phosphocreatine, pH 7.3, adjusted with 1 M NaOH, 280-290 mOsm) were used to record excitatory synaptic currents and action potential activity. The junction potential was not compensated for, and offset potentials were nulled before gigaseal formation. The resting membrane potential was measured immediately upon establishing whole cell configuration. Firing patterns of sympathetic neurons were determined in current-clamp mode immediately after achieving whole-cell configuration by a series of hyperpolarizing and depolarizing steps of current injection. For each neuron, the threshold for action potential generation was determined as the difference between the resting membrane potential and the membrane potential at which phase plot slope reached 10 mV/ms (35).

Mass Spectrometry of Brain Samples Mice were sacrificed 30 min post-injection with AMPH and PEGyAMPH (dose: 0.12 mol/kg of BW for both drugs, IP), brain samples were snap-frozen in liquid nitrogen before extraction procedures (36). Brain samples were smashed and extracted using ice-cold 1 mM perchloric acid (500 μL per sample) and left extracting overnight. After this time, the samples were centrifuged twice for 20 min at 5000 rpm, 4° C. Supernatants were transferred to new vials, frozen and freeze dried overnight of each time, concentrated up to 50 μL. Then, 25 μL of the remaining solutions were diluted in 75 μL of an electrospray ionization solution (ACN:H₂O in 3:1 ratio). Such mixtures were evaluated through direct injection using a FT-ICR mass spectrometer (Bruker Apex Ultra, 7 Tesla actively shielded magnet).

### High-Fat Diet Challenge and Treatment.

When mice reached 8 weeks of age, or 1 day after sympathectomy, normal diet was replaced with high fat diet (HFD, Ssniff, Spezialdiäten, Soest, Germany, D12492) concomitantly with treatment (PBS, AMPH or PEGyAMPH, dose: 0.12 mol/kg of BW for both drugs, daily IP injections). Length of exposure to HFD is indicated in figure legends.

### Blood and Plasma Analysis.

Blood was collected from the tail vain of HFD fed mice, 2 h post-injections with PBS, AMPH or PEGyAMPH, without access to food. Blood glucose was measured using a glucometer (Accu-Check, Roche). Analysis of Insulin, Triglycerides, Glycerol and FFA levels in plasma as performed using Mouse Ultrasensitive Insulin ELISA (Alpco), Triglyceride Quantification Kit (Abcam), Free Glycerol Reagent (Sigma) and Glycerol Standard Solution (Sigma), and Free Fatty Acid Quantification Kit (MAK044, Sigma), respectively according to manufacturer's instructions.

### Tissue NE Measurements (ELISA)

To assess peripheral NE content in tissues, mice were sacrificed in ad libitum conditions 2 h post injection with PBS, AMPH or PEGyAMPH. NE levels were determined with an NE ELISA kit (Labor Diagnostika Nord GmbH). Tissues were homogenized and sonicated in homogenization buffer (1 N HCl, 1 mM EDTA, 4 mM Sodium metabisulfite), and cellular debris were pelleted by centrifugation at 20,000 g for 10 min at 4° C.). All tissue samples were normalized to total tissue protein concentration.

### Faecal Output Assay

24 h faecal output was collected and weighed. The faeces were washed with 1×PBS and total triglyceride content was extracted by homogenization and boiling, for 2 cycles of 5 min, in 5% NP-40. Triglyceride content was measured using Triglyceride Quantification Kit (Abcam), according to manufacturer's instructions, and normalized to the weight of total faecal output.

### Tissue Triglycerides Analysis.

To assess muscle and liver content in tissues, mice were sacrificed in ad libitum conditions 2 h post injection with PBS, AMPH or PEGyAMPH. Triglyceride content was measured using Triglyceride Quantification Kit (Abcam), according to manufacturer's instructions. Tissue samples were normalized to total tissue protein concentration.

### Locomotion Assays.

After 3 weeks of HFD exposure and treatment, mice were either acclimated to tracking cages for 1 week before starting the 72 h locomotion measurements using the LabMaster tracking system (TSE Systems; Bad Homburg); or filmed for 20-30 min, with a ZEISS optics camera, 1 h post injection inside their normal housing cage, for assessment of total distance traveled. Footage-records were filtered using the video editor Avidemux (Avidemux 2.7.1) and 10 or 15 min distance computations were quantified using the TrackMate tracking plugin from Fiji (Fiji; Wisconsin-Madinson).

### Quantitative PCR.

For gene expression analysis mice were sacrificed in ad libitum conditions 2 h post injection with PBS, AMPH or PEGyAMPH, tissues were collected and immediately frozen. Total tissue RNA was extracted using PureLink RNA Mini Hit (Invitrogen) according to manufacturer's instructions, from which complementary DNA was reverse-transcribed using SuperScript II (Invitrogen) and random primers (Invitrogen). Quantitative PCR was performed using SYBR Green (Applied Biosystems) in ABI QuantStudio 7 (Applied Biosystems). Glyceraldehyde 3-phosphate dehydrogenase (GAPDH) was used as housekeeping gene to normalize liver and muscle tissue samples. Acidic ribosomal phosphoprotein P0 (Arbp0) was used as housekeeping gene to normalize adipose tissues samples.

### Thermoregulation Studies.

All measurements were done in ad libitum fed mice 2 h post-injections. Rectal temperature was measured with an electronic thermometer (Precision). BAT and Tail thermographic pictures were taken using a Compact-Infrared-Thermal-Imaging-Camera (FLIR; West Mailing) and FLIR-Tools-Software (FLIR; West Mailing) to quantify local temperatures.

### Histopathological Analyses.

Mouse tissues were fixed in buffered formalin, and inclusion in paraffin was done according to standard technical procedures. Histopathology studies were performed on formalin-fixed and paraffin-embedded sections of 3-6 μm thick for Haematoxylin and Eosin and for Oil-Red staining. Tissues were analysed using a Leica DM LB2 microscope, and images were captured with a Leica DFC 250 camera.

### Statistics.

Statistical analyses were performed using GraphPad Prism software (San Diego, Calif.) using unpaired Student's t-test (two-tailed) when two groups were being compared or one-way ANOVA test when several groups were being compared. One way-ANOVA was followed by Tukey's multiple comparison test or Bonferroni multiple comparison test with one group indicated as a control group. A P<0.05 was considered statistically significant. Data were represented as mean±SEM. Sample size was predetermined based on previous studies. Data displayed normal variance.

### Data Availability

The RNA-seq data sets are available at GEO accession code GSE103847.

### Results

### Specialized Morphology and Activation of SNS Cx3cr1⁺ Cells

Our initial aim was to visualize the in vivo morphology of ATMs using two-photon and confocal microscopy in Cx3cr1^{GFP/+} mice, in which macrophages are GFP-labelled. ATMs in fat parenchyma had a regular circular shape, whereas those located on sympathetic nerve bundles exhibited profuse pseudopodia that extended over greater surface area. Furthermore, we observed that sympathetic neuron-associated Cx3cr1^{GFP/+} cells displayed dynamic extensions and retractions of dendritiform processes over time. In contrast, ATMs surrounding adipocytes displayed minimal temporal plasticity or displacement. Using correlative light electron microscopy on WAT-derived nerve bundles, we confirmed that Cx3cr1^{GFP/+} cells extended thin pseudopodia processes that envelop non-myelinated SNS axons.

We then investigated whether sympathetic neuron-associated Cx3cr1^{GFP/+} cells were present in other SNS compartments, such as paravertebral sympathetic ganglia. Upon imaging superior cervical ganglia (SCG) and thoracic chains, we visualized Cx3cr1^{GFP/+} cells that were morphologically similar to those within WAT-derived SNS bundles. Due to established ex vivo explant potential, we used SCGs along with WAT-derived SNS nerve bundles as model systems for subsequent functional and molecular analyses.

### SNS Cx3cr1 SAMs Exhibit Hematopoietic Characteristics

Because nearly all Cx3cr1^{GFP/+} cells isolated from sympathetic fibres were positive for the immune marker CD45 and macrophage marker F4/80, we designate these cells sympathetic neuron-associated macrophages (SAMs). Given the specialized morphology and location of SAMs, we next explored how these cells compared to other tissue macrophages and brain microglia. We sorted F4/80+CD45+ double-positive cells from the following tissues: sympathetic ganglia (SAM ganglia), sympathetic nerve fibres from inguinal fat (SAM fibres), neighboring subcutaneous fat (sATM), visceral fat (vATM), spleen (SpM) and brain (microglia). The relative abundance of CD45highCx3cr1-GFP+ cells was nearly four times higher within nerve fibres (SAMs) than in sWAT. CD45 is highly expressed in hematopoietic cells but expressed at low levels in microglia. Flow cytometric analysis revealed that SAMs are CD45medium/high, suggesting a hematopoietic origin of these cells. To this end, we generated bone marrow chimeras from CD45.2+Cx3cr1GFP/+ donors into irradiated CD45.1 recipient mice and observed complete repopulation of CD45+ cells derived from Cx3cr1GFP/+CD45.2 donors. Eight weeks post-transplantation, we established that CD45.2+Cx3cr1GFP/+ SAMs repopulated sympathetic nerve bundles in WAT, whereas microglia repopulation in the brain did not occur. This suggests that SAMs in sympathetic fibres have similar origin to other hematopoietic macrophages as opposed to microglial lineage.

### SAM Expression Profile is More Macrophage-than Glia-Like

Given their association with neurons, we asked how the gene expression profile of SAMs compared to other resident tissue macrophages in microglia. We sorted macrophages from various tissues as described above (F4/80+CD45+ double-positive cells designated as SAM ganglia, SAM fibres, sATMs, vATMs, SpM, and microglia) and profiled gene expression by low input RNAseq. As expected, SAMs highly expressed markers common to both microglia and macrophages, such as Adgre1, Csf1r, Cx3cr1. SAMs expressed macrophage-associated genes that are excluded from microglia, such as Fn1 or Ciita¹². By flow cytometric analysis, additional macrophage-specific markers that are excluded from microglia (CD68, Ly6c, MHCII, and CD11b) were also highly expressed in SAMs. SAMs do not robustly express microglial-orglial-specific genes relative to macrophage-specific genes¹³⁻²². Sall1, a key microglia lineage-determining transcription factor, is strikingly absent from SAMs²³.

Principle component analysis (PCA) of the RNAseq data shows tight clustering across replicates, indicating low contamination and high reproducibility. The absence of tyrosine hydroxylase (Th) expression in SAMs further excluded the possibility of contaminating cargofrom neighbouring cells, as Th is highly expressed in adjacent SNS neurons. PCA analysis indicated that SAMs from fibres and ganglia are closely related, but both are distant from microglia and other macrophages. This is confirmed by phylogenetic analysis.

We hypothesized that the increased motility of SAMs could indicate an activated, pro-inflammatory state. Therefore, we measured expression of a constellation of pro- and anti-inflammatory markers in SAMs by RNA-seq. Relative to other macrophage populations, SAMs highly expressed genes associated with macrophage activation, including Cxcl2, Tnf, Socs3, and ll1a, suggesting a constitutively pro-inflammatory steady state.

SAMs are Phylogenically Distinct from Other Macrophages

Consistent with the PCA analysis, Pearson correlation analyses of transcript levels indicated differential expression patterns across SAMs, sATMs, vATMs, SpMs and microglia. Adipose tissue macrophages (sATMs and vATMs) showed similar expression landscapes (R=0.92) that are distant from fibre SAMs (R=0.63 for sATM and R=0.61 for vATMs. Microglia and spleen macrophages were least correlated with other groups.

Gene ontology analyses indicated several biological processes associated with genes enriched in SAMs relative to surrounding sATMs. SAMs preferentially expressed genes involved in synaptic signaling, cell-cell adhesion, and neuron development, suggesting that these cells fulfil an intrinsic role in local neuronal maintenance. Taken together, these data demonstrate divergent gene expression patterns in SAMs and ATMs, constituting intra-tissue macrophage specialization.

### SAMs Import and Degrade, but do not Synthesize, NE

We next examined the specific transcripts comprising divergent macrophage gene expression landscapes. The aforementioned populations of macrophages were sorted for transcriptome analysis via low-input RNA-seq. Given the gene ontology results and spatial proximity of SAMs to nerves, we hypothesized differential expression of neurotransmitter receptors, transporters or catalysing enzymes. Consistent with the ImmGen database, we detected abundant β2 adrenergic receptor (Adrb2) expression in all macrophage populations, which was confirmed by qRT-PCR.

However, SAMs were the only population that expressed Slc6a2, the gene for the NE transporter. Similarly, Maoa, the gene encoding MAOa, was highly expressed in SAMs relative to the other macrophage types. Both results were validated by qRT-PCR (Table 2). As Slc6a2 imports and MAOa degrades NE, we also tested for and detected NE by ELISA in sorted SAMs. Consistent with our results, neither Slc6a2 nor Maoa are significantly expressed in any macrophage population listed in the ImmGen database. Furthermore, we validated Slc6a2 and MAOa protein expression by immunofluorescence in Cx3cr1GFP/+ SNS nerve fibres and SCG cryo-sections. Representative photomicrographs depict GFP containing SAMs were double-positive for membrane-bound Slc6a2 or mitochondrial-bound MAOa.

As SAMs, but not other macrophage types assessed, possess the molecular machinery for import and degradation of NE, as well as significantly more NE relative to other macrophages, we tested the possibility that SAMs synthesize NE. By qRT-PCR of sorted SAMs, we did not detect expression of Th, which encodes an enzyme necessary for NE biosynthesis. Taken together, these results indicate that SAMs possess the molecular machinery for importing and degrading NE, but not for biosynthesis.

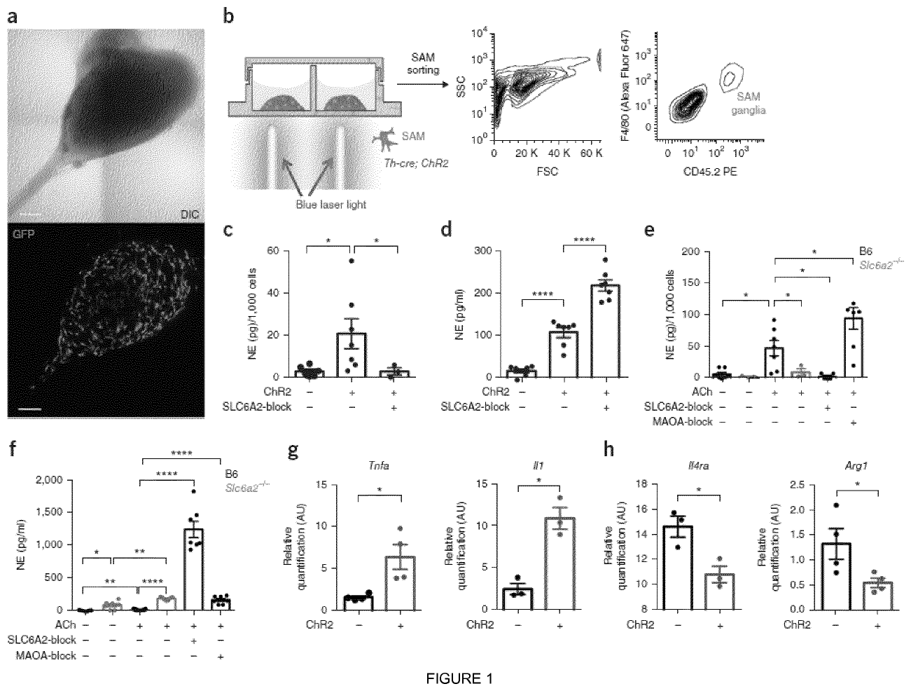

To explore the responsiveness of SAMs to NE, we optogenetically stimulated sympathetic neurons in SCG cultures from TH-Cre X Rosa26-LSL-ChR2-YFP mice, which allowed us to visualize sympathetic neuron-macrophage interactions ex vivo (FIG. 1a,b). After optogenetic stimulation, we measured NE content of sorted CD45+F4/80+ cells. SAMs from ChR2-positive cultures exhibited significantly higher NE levels (FIG. 1c) that were proportional to NE availability in the culture medium (FIG. 1d). NE release by ChR2-positive neurons was significantly higher relative to ChR2-negative neurons (FIG. 1d). Uptake of NE by SAMs was prevented by pharmacologic blockade of Slc6a2 by the pharmacological inhibitor Nisoxetine, despite significant increase of NE in the culture medium (FIG. 1c,d).

To validate our optogenetic findings with a physiologically relevant stimulus, we activated SNS explants with acetylcholine (ACh), which is pre synaptically released from spinal cord neurons to innervate ACG. ACh-treated CD45+F4/80+ cells sorted from SCG explants contained significantly higher levels of NE than vehicle controls (FIG. 1e). We validated that blockade of the NE importer Slc6a2 by Nisoxetine prevented NE accumulation in SAMs (FIG. 1e). Co-incubation with ACh and Nisoxetine further abolished NE uptake (FIG. 1e) despite the substantial increase of extracellular NE levels in the culture medium (FIG. 1f). These results, along with the negligible expression levels AChRs in SAMs (also validated by qRT-PCR), excluded a role for AChRs in mediating NE import.

Next, we assessed the effect of blocking MAOa on NE content in CD45+F4/80+ cells (FIG. 1e). The MAOa inhibitor clorgyline was sufficient to nearly double intracellular NE levels in SAMs (FIG. 1e). Consistently, clorgyline increased NE levels in medium (FIG. 1f), to which neuronal MAOa expression may also contribute. Genetic ablation of Slc6a2 (using SCG isolated from Slc6a2−/− mice) prevented NE uptake by SAMs regardless of the NE availability in the culture medium (FIG. 1e,f). Finally, ATMs cultured in vitro with NE did not accumulate intracellular NE, further demonstrating the specificity of NE uptake by SAMs. Altogether, our results indicate that Slc6a2 is required for NE accumulation in SAMs.

We further probed whether the availability of NE, which can be manipulated in vivo by optogenetic activation of SNS neurons, changes the inflammatory profile of SAMs. We found that optogenetic stimulation of SCG explants correlated with an increase of pro-inflammatory gene expression as measured by changes in Tnfa and 111 (FIG. 1g) and decrease of anti-inflammatory gene expression as measured by changes in 114ra and Arg1 (FIG. 1h).

### SAMs are Recruited and Activated in Obesity

We next utilized two mouse models to characterize the effect of obesity on tissue-specific functions of SAMs. In total, we employed four experimental groups: high-fat diet (HFD)-fed, leptin-deficient (ob/ob), normal diet (ND)-fed, and 24-hr fasted ND-fed mice. Flow cytometric analysis demonstrated that both obesity models (HFD and ob/ob) exhibited significantly higher percentages of SAMs compared to lean mice (ND) (FIG. 2a). Furthermore, the acute metabolic challenge of fasting did not result in upregulation of SAMs, suggesting an obesity specific causation of elevated macrophage content in sympathetic fibres (FIG. 2a).

Within the F4/80+ SAM fraction in HFD and ob/ob mice, we noted a high frequency of CD11c+ cells (FIG. 2b), which are hallmarks of inflammation and insulin resistance in human obesity¹⁹. In contrast to SAM accumulation in SNS nerve fibres dissected from WAT, SAMs do not accumulate in SCG, which innervates neck structures such as salivary glands.

The differential distribution of macrophages in states of obesity suggested cytokine levels were also sensitive to obesity. Comparing anti- and pro-inflammatory gene profiles of SAMs, ATMs, and SpMs (FIG. 2c-e) revealed that obesity correlated with higher levels of pro-inflammatory gene expression (i.e., Tnfa or 111; FIG. 2c,e) and lower levels of anti-inflammatory gene expression (i.e., Arg1 or 1110; FIG. 2d,e).

To determine if local proliferation contributes to SAM accumulation, we measured the proliferation marker Ki67 in SAMs by flow cytometry. We observed that obesity (via HFD or ob/ob models) does not substantially increase Ki67+ SAM percentage, whereas (consistent with previous reports²⁵) obesity increases Ki67+ ATMs from sWAT.

Slc6a2 deletion in SAMs rescues obesity

We probed how ablating Slc6a2 in SAMs affected obesity associated pathology. We considered a Cre-Lox approach, but the established macrophage Cre lines (Cx3Cr1-Cre^{26,27} and LyzM-Cre²⁸) would not allow for SAM-specificity. We thus took advantage of the cell type-specificity of Slc6a2 expression, which is high in SAMs and negligible in other macrophage and hematopoietic populations (ImmGen²⁹). We validated that, besides SAMs, there did not exist another hematopoietic-derived population that expressed Slc6a2; a rare population of CD45+F4/80-cells were present in SCG but did not express Slc6a2. SAM-specific genetic ablation of Slc6a2 was attained by bone marrow transfer from Slc6a2−/− mice³⁰ into genetically obese ob/ob recipients (ob/obSlc6a2−/−) (FIG. 3a). Control chimeras consisted of bone marrow transfer from B6-CD45.1 mice into ob/ob recipients (ob/obCtrl). Chimeras recovered for nine weeks post-transplant to allow irradiation-induced inflammation to subside. As cold temperature is a robust driver of SNS activity, we challenged mice for 2 hr at 4° C. and observed that ob/obSlc6a2−/− chimeras displayed superior capacity for maintaining body temperature compared to control ob/obCtrl chimeras (FIG. 3b). These thermogenic effects were accompanied by significant upregulation of NE serum levels (FIG. 3c), rescue of BAT morphology (FIG. 3d), and browning of white fat, as measured by Ucp1 mRNA and protein levels (FIG. 3e-g).

Transplant with bone marrow from Slc6a2−/− into ob/ob mice prevented obesity-induced hypertrophy of both BAT and WAT adipocytes (FIG. 3h) but did not affect total body weight (FIG. 3i). Because food restriction challenge drives SNS activity and mobilizes lipid stores from adipose tissue, we normalized daily food intake of the ob/ob chimeras for 2 weeks (FIG. 3i,j). After a dieting challenge ob/obsic6a2−/− mice, relative to control chimeras, lost nearly 30% of body weight, which was stable up to 16 weeks, even after ad libitum access to food (FIG. 3i). Ob/obslc6a2−/− mice also exhibited higher lipid mobilization during food restriction (FIG. 3j).

We analyzed wild-type B6 chimeras reconstituted with control CD45.1 bone marrow or Slc6a2−/− bone marrow. SAMs from B6slc6a2−/− chimeras did not accumulate NE. Consistent with the results from ob/ob chimeras (FIG. 3), B6slc6a2−/− chimeras also exhibited increased serum NE levels, thermogenesis, and lipolysis, as well as marked weight loss, relative to B6ctrl mice. Upon HFD challenge, we observed weight gain prevention in B6slc6a2−/− but not in B6ctrl mice. These results indicate a significant anti-obesity effect of SAM-specific Slc6a2 ablation.

### SAMs are in BAT and Act as an NE Sink

In light of the enhanced thermogenic capacity of ob/obslc6a2−/− chimeras, we questioned if SAMs are present in BAT. BAT did contain Cx3Cr1GFP cells (consistent with previous reports²⁴) that exhibited an intermediate morphology between SAMs (multiple pseudopodia) and ATMs (round). Some of these cells appeared to make close contact with thin TH+ axons. Because TH+ nerve fibres in BAT are too delicate for dissection, we sorted macrophages from whole BAT for qRTPCR analysis. Slc6a2 and MAOa were expressed in BAT macrophages, although at lower levels relative to SAMs isolated from dissected SNS nerve bundles in sWAT or SCG. BAT macrophages also contained NE, although at lower levels than SAMs. The lower levels of Slc6a2, MAOa, and NE content may reflect a dilution of BAT-SAMs by BAT-ATMs since mixed (as opposed to isolated) populations were analyzed.

Finally, we used conditional LyzM-Cre; CSF1R-LSL-DTR mice to test if macrophages served as a sink for NE. After validating ablation of macrophages, we observed a significant increase of NE in sWAT in vivo. Note that, due to constant hematopoietic input, it is practically impossible to completely deplete all macrophages. This limitation notwithstanding, these results are consistent with a model in which macrophages act as sink for NE.

### Human Sympathetic Ganglia Also Contain NE-Degrading SAMs

Finally, we asked if SAMs exist in humans. We obtained nine human excisional biopsies of SNS or thoracolumbar ganglia that were collected during sympathectomy and/or gangliotomy. We stained tissue sections with H&E or an antibody against CD68, a human macrophage marker, identifying the presence of macrophages in SNS tissues.

We next determined whether SAMs in human sympathetic ganglia also contain the machinery for uptake and degradation of NE. The CD68 macrophage marker co-localized with staining for Slc6a2 and MAOa. Both Slc6a2- and MAOapositive neurons exist, but the background levels are low relative to control human gut-associated lymphoid tissue (GALT) samples that also contain CD68+ macrophages.

SAMs are a previously undescribed population of resident macrophages in the SNS that import and degrade NE. To fulfil their function, SAMs express a dedicated molecular machinery that is, as best we can tell, absent from neighbouring macrophages and other known macrophage populations (shown by our data and ImmGen database). In SAMs, NE is imported by Slc6a2 and degraded by MAOa. This is a specialized molecular mechanism for NE uptake, the role for which is not fulfilled by canonical phagocytic mechanisms generally present in macrophages³¹. Unlike most other neurons, which exclusively release neurotransmitter at a terminal synapse, SNS neurons also release NE via varicosities distributed along axons that can extend for tens of centimeters³². SAMs possibly serve to prevent NE spillover into the blood stream or neighbouring tissues during high SNS activity. Indeed, we demonstrate that when SNS neurons are optogenetically activated, SAMs import increased levels of NE and become more polarized towards a pro-inflammatory phenotype. In this regard, NE can be considered a noxious stimulus that must be locally delivered in a controlled manner to a target tissue. Chronic and excessive systemic NE in serum, such as in chronic stress conditions or medullary adrenal tumors, leads to hypertension and cardiopathy due to direct action in cardiovascular tissues³³.

The activated polarization state of SAMs is consistent with a model in which these cells play a tissue-protective role by acting as a sentinel and scavenger of excess levels of an endogenous neurotransmitter (i.e., NE) that, if released in excess from varicosities, could potentially be harmful. Tissue-protective immune cells have been documented in the brain and other non-neuronal systems³⁴⁻³⁸. For instance, muscularis resident macrophages in the gut induce rapid tissue-protective responses to potentially pathogenic insults via the β2-AR signaling³⁹. This and our study indicate specialization of macrophage populations to fulfil tissue-specific tasks in response to neuronal cues. Divergent gene expression landscapes across resident macrophage populations isolated from different tissues support the idea of local macrophage adaptations^{26,40, 41}. In this study, we use transcriptional data to molecularly characterize SAMs alongside other macrophage populations. Our results suggest that macrophages associated with the SNS have specialized molecular programs whose exploration might give further insight into mechanisms underlying SNS macrophage-neuron communication. Although SAMs express common microglia genes and reside in proximity to nerve cells, SAM pseudopodia are morphologically distinct from the finely branching ramifications of resting microglia^{42,43} Moreover, SAMs are seemingly of hematopoietic origin, as suggested by our bone marrow chimera studies and high expression of CD45 and macrophage markers. Future tracing studies are necessary to definitively determine SAM origin. No reports exist on NE uptake by microglia, and we verified that machinery for NE uptake is not expressed in these cells. In this regard, only one study has reported that NE can trigger microglia to import and degrade amyloid, but not NE itself⁴⁴. Neurotransmitter uptake has primarily been studied in astroglia, which are Cx3cr1-negative⁴⁵. Chimeric models require irradiation that generates inflammation. However, if given adequate recovery time (8 weeks), recruited macrophages dissipate from the brain, as represented in our chimeras by minimal residual Cx3CR1-GFP+ microglia (0.06%). SAM levels persist at levels that greatly surmount background irradiation-induced macrophage recruitment, and regenerated SAMs are seemingly identical to those in non-irradiated mice.

We show low expression of several astroglial markers in SAMs, raising the possibility of a hybrid peripheral cell type that unites some of the features of macrophages and glia. Alternatively, mutual genes of glial cells and SAMs may be attributable to their proximity to neuron derived signals, analogous to the observation that microglia, astrocytes and neurons share certain CNS specific genes^{11,46}. An alternative model is that SAMs share the lineage of satellite glial cells (SGC), which are derived from embryonic neural crest₁₁ and also express canonical astroglial markers⁴⁷. However, SGC import or degradation of NE has not been reported⁴⁸. Our study may fill a gap in the literature by demonstrating a cellular and molecular mechanism alternate to the proposed existence of NE-producing macrophages in WAT³. In this regard, our findings are consistent with other reports⁴⁻⁶, as we do not detect the NE biosynthetic machinery in SAMs nor in ATMs. The identification of SAMs sheds new light on this recent controversy by documenting how a particular population of macrophages can contain NE in the absence of its biosynthesis. We also document that BAT macrophages contain similar molecular machinery as SAMs for NE uptake, extending and validating the findings of our colleagues²¹. SAMs may play a tissue protective role by regulating regional NE levels by serving as a local sink that prevents the dangerous effects of chronically increased levels of systemic NE. In sharp contrast to the anti-inflammatory state of intestinal nerve-associated Cx3Cr1GFP macrophages⁴⁹, SAMs exhibit a pro-inflammatory profile at steady state. This could be due to the constitutive presence of a danger signal—namely, NE. Whether the polarization is caused by NE import or by adrenergic signalling remains to be established. In this regard, polarization of enteric-associated macrophages has been linked to activation of beta-2 adrenergic receptor, which is also expressed in SAMs⁴⁹. Regardless, our core message is relevant: that SAMs are pro-inflammatory and act as an NE sink and that blocking NE uptake has an anti-obesity effect. Our results support a model whereby SAMs pathologically accumulate in SNS nerves of obese subjects in an organ-specific manner, thus explaining why we detect SAM accumulation in the WAT²⁶ associated SNS, but not in SCG, which innervates salivary glands and other neck structures. The NE scavenging role of SAMs may have become evolutionarily maladaptive, as, in the past, obesity was not a common physiological stress to which humans had to adapt. In modern times, the prevalence of over nutrition has necessitated a need for increased lipolysis-inducing NE signalling to maintain fat stores, which is obstructed by the “original” function of SAMs to limit NE levels. Reduced NE availability in the adipose tissue is linked to blunted lipolysis and obesity. Very recently, our colleagues have shown that ATMs degrade NE during ageing⁵⁰. Whether this observation is also associated with SAMs accumulation in the fat, as we observe in two mouse models of obesity, remains to be established. Our results demonstrate that SAM specific Slc6a2 ablation rescues BAT and adaptive thermogenesis in obese ob/ob mice, which in turn leads to sustained weight loss and lipid mobilization. We determine that blocking NE import into SAMs mitigates the recidivism of obesity that is typical after dieting. Overall, our results identify SAMs as a potential new molecular and cellular target for obesity therapy.

Amphetamine blocks Slc6a2 (NET, norepinephrine transporter) and is a potent anti-obesity agent. Our results discussed herein establish that loss of function of Slc6a2 from the hematopoietic compartment has an anti-obesity effect. This led us to hypothesize a new mechanism of action by which Amphetamine promotes weight loss and fat mass reduction independently of an action in the brain. This hypothesis challenges the classic textbook model that AMPH is a potent anti-obesity drug because it acts in the brain to promote satiety and excessive locomotion (hyperkinesia).

### The Sympathomimetic Activity of AMPH is Required for its Anti-Obesity Effect.

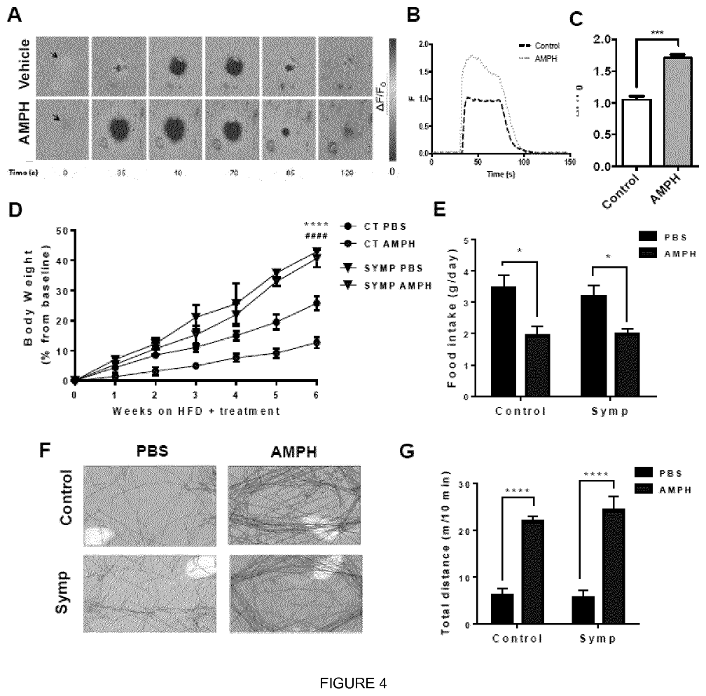

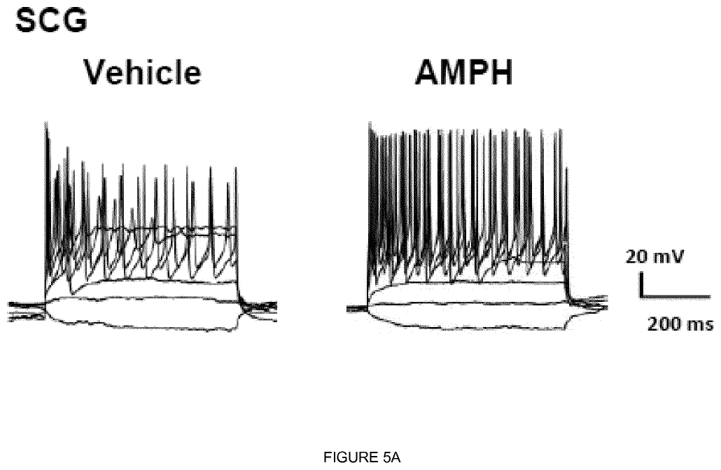

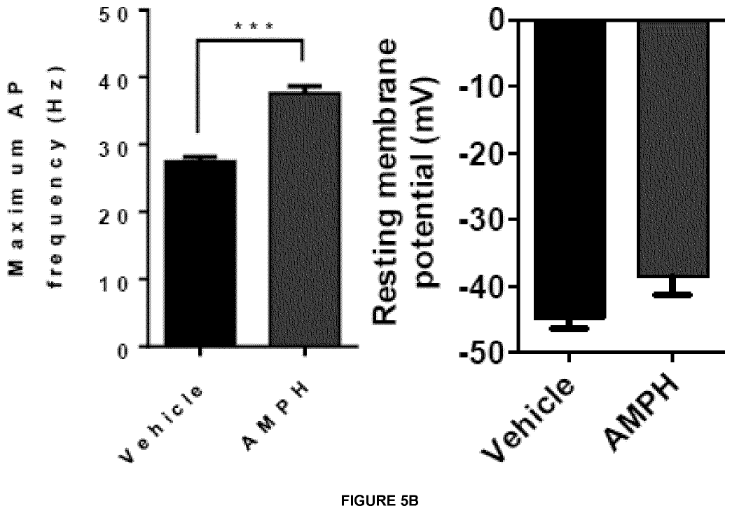

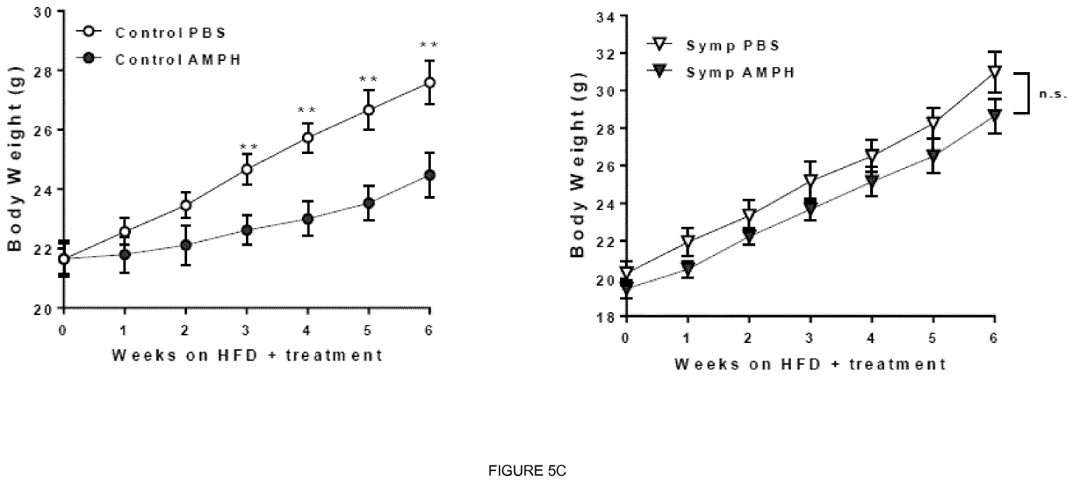

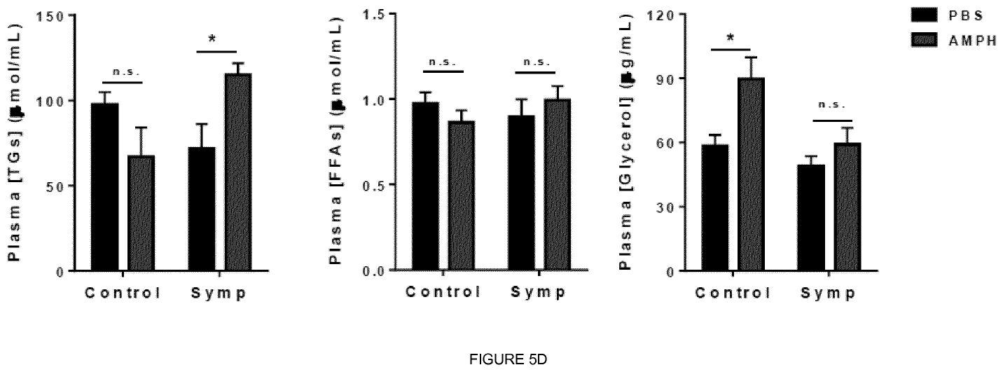

We probed AMPH's effect on excitability of sympathetic neurons isolated from superior cervical ganglia (SCG), by using calcium imaging as well as electrophysiology. For calcium imaging we used dissociated cultures of TH-cre; CAG-LSL-GCaMP3 (GCaMP3⁺) reporter mice. Local application of Acetylcholine (ACh), a physiologic pre-ganglionic activator, increased the intracellular [Ca²⁺] in sympathetic neurons from GCaMP3⁺ mice in control experiments (Vehicle) by 1.05±0.05. Neurons treated with AMPH have significantly higher increase of the ΔF/F₀ to 1.71±0.05 (p<0.001—FIGS. 4A-C). In parallel, we recorded firing patterns of wild type neurons isolated from C57BL/6 mice, by whole cell patch-clamp recordings under current-clamp mode, and observed that AMPH significantly increases the maximum firing frequency (27.48±0.72 Hz in Vehicle, 37.60±1.07 Hz in AMPH-treated neurons, p<0.001, FIGS. 5A, B), while no significant changes in resting membrane potential were observed (FIGS. 5A, B). These results demonstrate that AMPH treatment increases the intrinsic excitability of peripheral sympathetic neurons.

To investigate whether the increase in peripheral adrenergic signalling is required to the anti-obesity effect of AMPH, we subjected LSL-DTR (Control) and sympathectomized⁵¹, TH-cre; LSL-DTR mice (Symp mice) to an obesogenic high fat diet (HFD) accompanied of AMPH treatment (0.12 mol/kg of BW, or control PBS, daily intraperitoneal (IP) injections) for a total of 6 weeks, and assessed body weight-gain over time. As expected, AMPH treatment protects control mice from diet induced obesity (DIO) (25.75±2.34% of BW gain for PBS treated vs 12.67±1.79%); AMPH treated control mice (circular data points, p<0.01—FIG. 4D). As previously reported (Pereira, M. M. A. et al. Nat. Commun. 8, 14967 (2017)), Symp mice become extremely prone to DIO and gain twice as much weight as the Control group after 6 weeks of HFD exposure (44.55±6.55% of BW gain for PBS treated Symp mice, white triangular vs circular data points, p<0.0001—FIG. 4D). Surprisingly, both cohorts of Symp mice had very similar BW-gain rate upon HFD exposure, regardless of treatment, leading to about 40% increase after just 6 weeks (39.19±4.54% of BW gain for AMPH treated Symp mice—triangular data points, FIGS. 4D and 5C). This phenotype was independent from behaviour (FIGS. 4E-G): both Control and Symp groups showed significant reduction in food intake (PBS treated groups: 3.63±0.35 g/day for Control mice and 3.12±0.31 g/day for Symp mice vs AMPH treated groups: 2.08±0.25 g/day and 2.00±0.12 g/day, respectively, p<0.01—FIG. 4E) and increase in locomotor activity (PBS treated groups: 6.26±1.39 m, during 10-min video-tracking, for Control mice and 5.55±1.69 m for Symp mice, vs AMPH treated groups: 21.88±1.09 m and 24.30±2.88 m, respectively, p<0.0001—FIGS. 4F, 4G) with AMPH treatment. We hypothesised that underlying this phenotype was a reduction in sympathetic output (NE levels) to white adipose tissue (WAT). To assess this, we measured NE content in inguinal WAT of AMPH-treated mice and noted a marked reduction in Symp relative to Controls (PBS treated groups: 1.73±0.19 ng/mg of total tissue protein in Control mice, and 1.23±0.14 ng/mg in Symp mice; vs AMPH treated groups: 2.58±0.28 ng/mg and 1.51±0.20 ng/mg, respectively: p<0.05 only between Control mice groups). We also analysed plasma lipid content 2 h post-injection (Glycerol levels on the right—PBS treated groups: 58.42±5.05 μg/mL in Control mice and 48.95±4.56 μg/mL in Symp mice vs AMPH treated groups: 89.70±10.20 μg/mL and 59.07±7.83 μg/mL, respectively, p<0.05 only between Control mice groups— FIG. 5D) to evaluate the levels of adrenergic-stimulated lipolysis, which might explain the necessity of an intact SNS. In fact, in Symp mice, the behavioural effects of AMPH were not accompanied by the increase in SNS tone neither the elevation of lipolysis as observed in Control AMPH treated mice (FIG. 5D). These results establish that the sympathomimetic activity of AMPH is required for its protection against weight gain. More importantly, the finding that the reduced food intake and increased locomotion observed in AMPH treated Symp mice were not much effective in reducing their BW-gain rate in the absence of a functional SNS. SNS is thus a direct and necessary target of AMPH that mediates its anti-obesity effect, independently of hypophagia and hyperkinesia and activation of SNS by AMPH upregulates lipolysis in vivo.

PEGylation of AMPH Retains Peripheral Sympathomimetic Activity and Prevents its Access to the Brain without Affecting Behaviour

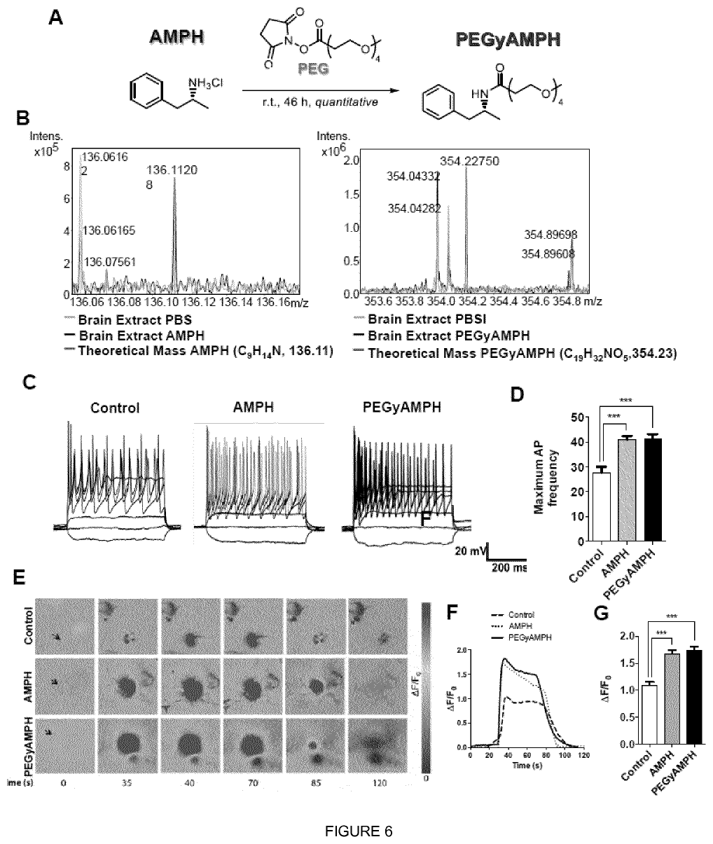

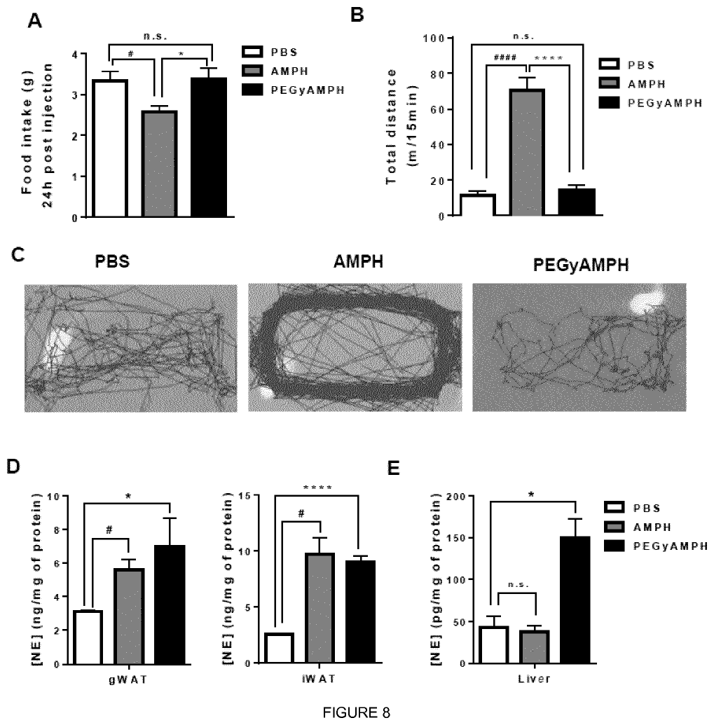

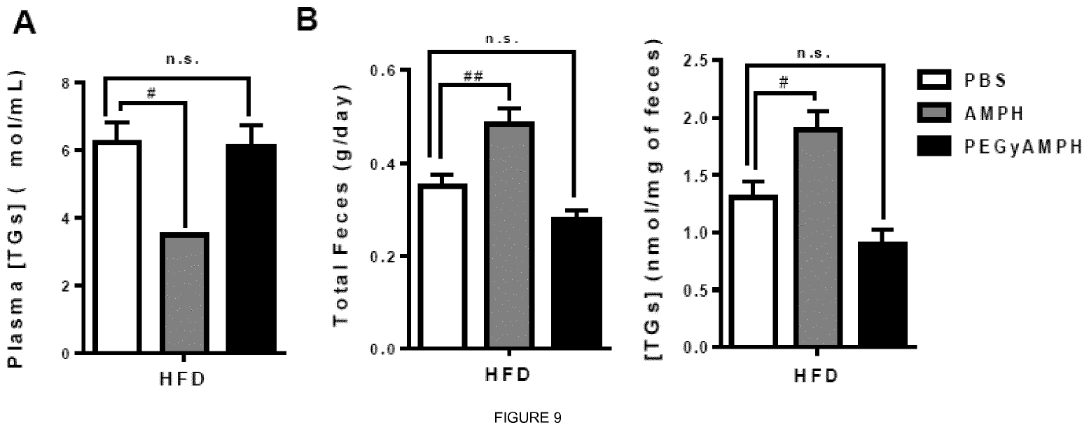

Big molecules are generally impermeable to the blood-brain-barrier, thus we employed PEGylation to increase the size of AMPH, herein named PEGyAMPH (FIG. 6A). We injected (0.12 mol/kg of BW for both drugs, or control PBS, IP) wild type adult C57BL/6 mice with AMPH or PEGyAMPH and collected brains 30 min afterwards, considering that the half-life AMPH in mice is reported to be about 20-50 min⁹. Brain extracts were analysed by mass-spectrometry to detect the presence of either molecules (FIG. 6B). Given the high resolution conferred by the FT-ICR, one can identify the compound with errors lower than 1.5 ppm, from the all replicate brain samples. Only in the group treated with AMPH was the drug detectable 30 min post-injection (FIG. 6B). We also assessed earlier time points (5 min, 1 h and 2 h post-injection) but PEGyAMPH is never detected in the brain. We then probed behavioural alterations in mice immediately after injection of either drugs (FIG. 8). According to the previous results, AMPH treatment alters feeding behaviour (3.34±0.24 g, 24 h post-injection, for PBS treated mice; 2.57±0.15 g for AMPH treated mice, (red) p<0.05— FIG. 8A) and locomotor activity in mice (11.34±2.23 m, during 15-min video-tracking, for PBS treated mice; 70.45±7.54 m for AMPH treated mice, p<0.0001— FIGS. 8B, 8C). However, we did not observe any significant changes in food intake (3.39±0.27 g for PEGyAMPH treated mice (dark)— FIG. 8A) nor locomotion (14.15±2.87 m for PEGyAMPH treated mice—FIGS. 8B, 8C) in PEGyAMPH injected mice compared to the control PBS group. Furthermore, the effects of AMPH on the gastrointestinal tract⁵² are absent when PEGyAMPH is administered. We probed dietary absorption during HFD feeding and found that PEGyAMPH administration did not alter the total 24 h faecal output of C57BL/6 mice, nor its lipid content (total faeces (left): PBS—0.35±0.03 g, AMPH—0.48±0.03 g, PEGyAMPH—0.28±0.02 g; triglycerides (TGs) levels (right): PBS—1.30±0.14 nmol/mg of faeces; AMPH—1.89±0.15 nmol/mg; PEGyAMPH—0.89±0.13 nmol/mg; FIG. 9B). Plasma TGs levels of PEGyAMPH injected mice were also unchanged compared to those of control mice in the fed-state, 2 h post-injection without access to food (PBS—6.22±0.60 μmol/mL; AMPH—3.48±0.01 μmol/mL; PEGyAMPH—6.09±0.66 μmol/mL—FIG. 9A). These results confirm that, unlike PEGyAMPH, AMPH not only reduces food intake and increases locomotor activity, but also increases faecal output via increased TG expulsion in faeces.

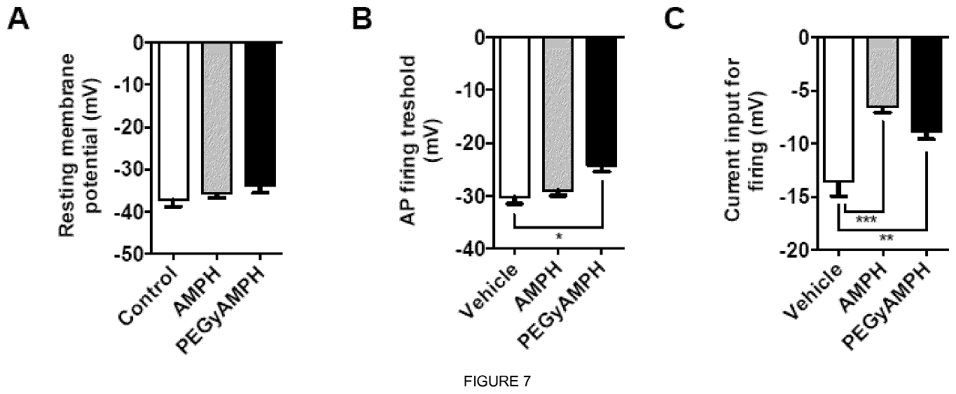

Next, to evaluate any loss of potency that might occur after PEGylation, we ascertained whether PEGyAMPH retains the ability to increase the excitability of sympathetic neurons. As aforementioned, we cultured and treated SCG neurons with either drugs and started by recording the firing patterns of sympathetic neurons, by performing whole cell patch-clamp recordings under current-clamp mode (FIGS. 6C-D; FIG. 7). The maximum firing frequency of PEGyAMPH-treated neurons significantly increased compared to control (27.71±2.37 Hz vs 41.00±1.43 Hz in AMPH-treated neurons and 41.29±1.93 Hz in PEGyAMPH-treated neurons, p<0.001, FIG. 6D). No significant changes in resting membrane potential were observed (−37.23±1.60 mV in Vehicle, −35.70±1.02 mV in AMPH-treated neurons and −33.21±1.59 mV in PEGyAMPH-treated neurons, FIG. 7A) and a significant increase in action potential firing threshold were observed only between vehicle and PEGyAMPH-treated neurons (−30.23±1.22 mV and −24.15±1.24 mV, respectively, p<0.05—FIG. 7B). It was also observed a significant decrease in the current input for firing (−13.61±1.35 mV in Vehicle, −6.55±0.49 mV in AMPH-treated neurons and −8.86±0.72 mV in PEGyAMPH-treated neurons, p<0.05—FIG. 7C). When we assessed PEGyAMPH's effects on intracellular [Ca²⁺] of sympathetic neurons isolated from GCaMP3+ reporter mice. After local application of ACh, there was a significant increase of ΔF/F₀ after incubation with PEGyAMPH when compared with control values, similarly to what was observed in AMPH-treated sympathetic neurons (1.09±0.06 in Vehicle and 1.74±0.06 in PEGyAMPH-treated neurons, p<0.001—FIGS. 6E-G). When tested in vivo, administration of PEGyAMPH, like AMPH (0.12 mol/kg of BW for both drugs and control PBS, IP), elevates peripheral sympathetic tone to adipose tissue. This was probed by the quantification of NE content in both gonadal WAT (gWAT) and iWAT 2 h post-injection (in gWAT (left): PBS—3.13±0.07 ng/mg of total tissue protein—vs AMPH—6.63±0.58 ng/mg—p<0.05; PBS vs PEGyAMPH—6.99±1.68 ng/mg—p<0.05; in iWAT (right): PBS—2.54±0.13 ng/mg vs AMPH—9.69±1.49 ng/mg—p<0.05; PBS vs PEGyAMPH—9.05±0.5 ng/mg— p<0.000, FIG. 8 D, 8E). These results confirm that PEGyAMPH is a peripheral sympathomimetic drug that elevetaes NE content in WAT without entering the brain and inducing behavioural changes.

PEGyAMPH Protects Mice from Obesity.

To investigate whether the increase in SNS activity would be sufficient to protect mice against obesity, treated adult wild-type C57BL/6 mice under HFD with either AMPH or PEGyAMPH (0.12 mol/kg of BW for both drugs, and control PBS, daily IP injections) for a total 10 weeks, and subsequently assessed their rate of weight gain and metabolic alterations.

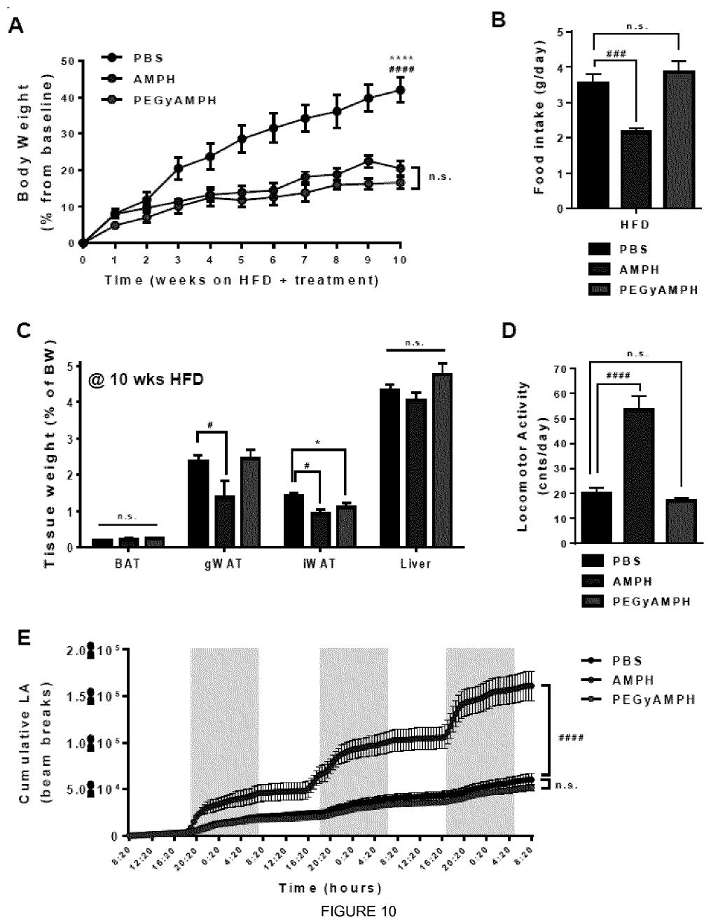

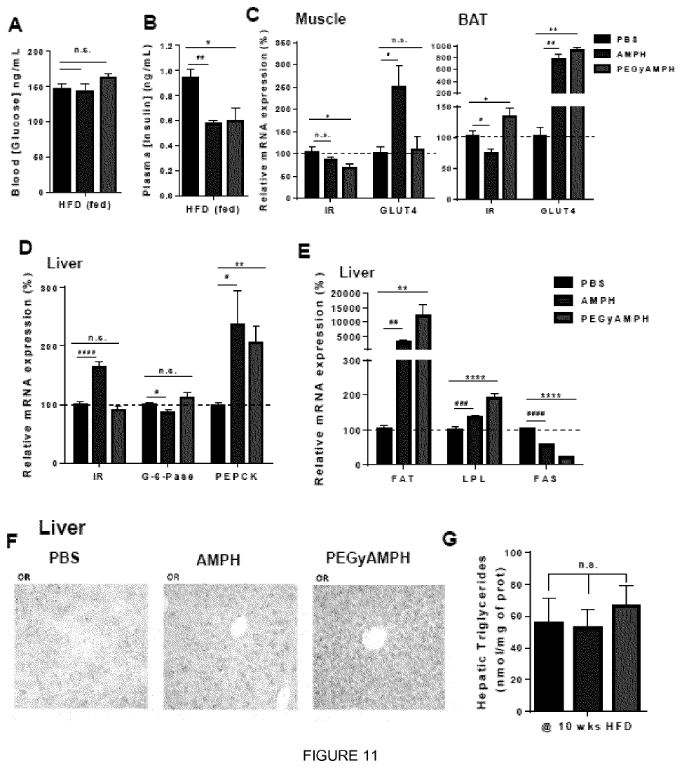

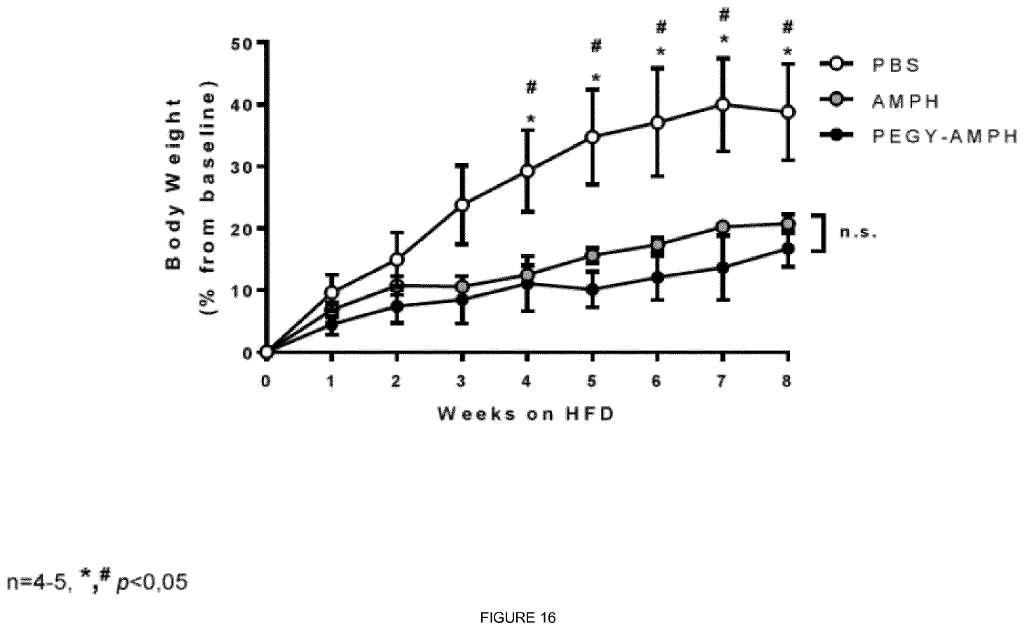

As demonstrated above, AMPH therapy protects wild-type mice from DIO (41.99±3.43% of BW gain, after 10 weeks of HFD, in PBS treated mice; 20.49±2.10% in AMPH treated mice, p<0.0001—FIGS. 10A and 16, red data points). Notably, treatment with PEGyAMPH showed similar size effect on body weight (16.58±1.70% of BW gain in PEGyAMPH treated mice, p<0.0001—FIGS. 10A and 16, blue data points). This reduction in body weight gain, was specifically associated to lower levels of adiposity compared to PBS-treated group after the 10 weeks of HFD exposure and treatments (iWAT: PBS—1.40±0.11% of total BW; AMPH—0.92±0.13%; PEGyAMPH—1.09±0.11%, p<0.05—FIG. 10C), without affecting the size of BAT or Liver (FIG. 10C). In fact, as expected, PEGyAMPH-treated mice do not decrease daily food intake (PBS— 3.58±0.25 g/day; AMPH—2.17±0.09 g/day; PEGyAMPH—3.85±0.32 g/day—FIG. 10B) nor elevate of locomotor activity (PBS—20.10±2.01 (a.u.) counts/day; AMPH—53.72±5.27 counts/day; PEGyAMPH—17.12±1.14 counts/day—FIGS. 10D, 10E) during treatment. Moreover, both therapies improved peripheral insulin sensitivity, which do not differ between all the HFD exposed groups (PBS—145.60±7.30 ng/mL in fed-state, 2 h post-injection without access to food; AMPH—142.50±10.48 ng/mL; PEGyAMPH—161.75±6.52 ng/mL—FIG. 11A). Circulating plasma insulin levels are significantly lower than those of the control PBS-treated mice (PBS—0.947±0.063 ng/mL in fed-state, 2 h post-injection without access to food; AMPH—0.582±0.020 ng/mL; PEGyAMPH—0.594±0.111 ng/mL p<0.05—FIG. 11B). In fact, the higher insulin sensitivity of the PEGyAMPH group was associated with a strong increase in the levels of mRNA expression of the insulin-dependent Glucose-Transporter-type-4 isoform (GLUT4) in BAT (FIG. 11C), but not in the muscle (FIG. 11C), as it is observed in the AMPH treated animals—probably due to increased exercise. Quantification of gene expression showed that both treatments also alter liver glucose metabolism (FIG. 11D). We observed no evidence of fatty liver assessed by Oil-Red lipid histology of liver slices, even in PBS treated mice, after 10 weeks of HFD exposure (FIG. 11F).

PEGyAMPH Protects from Obesity by Elevating Lipolysis.

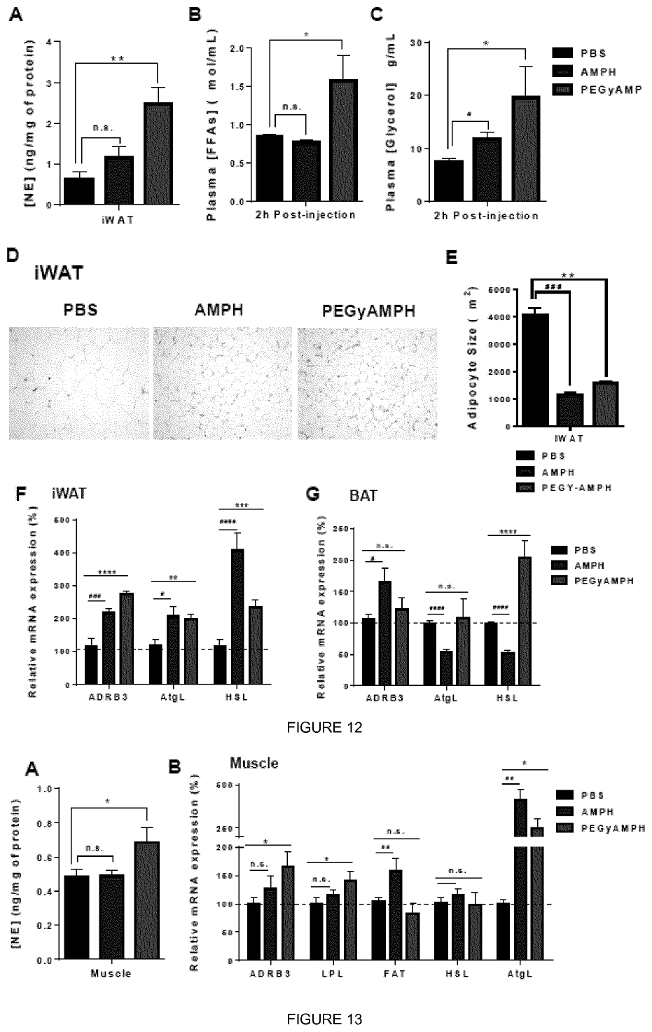

Next, as PEGyAMPH acts as a peripheral sympathomimetic, we hypothesised that treatment would affect adipose tissue physiology by increasing adrenergic-stimulated metabolic pathways, namely lipolysis and non-shivering thermogenesis, protecting mice from DIO. We started by confirming the increase in SNS activity in adipose tissue, by quantifying NE levels in iWAT of C57BL/6 mice. We found that, after 10 weeks of HFD exposure and treatment, the PEGyAMPH group had a superior increase of NE content in the iWAT (PBS—0.615±0.199 ng/mg of total protein; AMPH—1.166±0.263 ng/mg; PEGyAMPH—2.478±0.413 ng/mg; p<0.01 for PBS vs PEGyAMPH treatment—FIG. 12A), indicating that treatment with PEGyAMPH had higher sympathomimetic potency than the unmodified AMPH. This effect aligned with a peripherally acting drug with altered biodistribution and increased stability conferred by pegylation. NE levels were elevated also in the liver (PBS—1.387±0.136 ng/mg of total protein; AMPH—1.327±0.262 ng/mg; PEGyAMPH—2.09±0.306 ng/mg; p<0.05 for PBS vs PEGyAMPH treatment) and in the muscle (PBS— 0.484±0.041 ng/mg of total protein; AMPH—0.493±0.030 ng/mg; PEGyAMPH—0.686±0.085 ng/mg, p<0.05 for PBS vs PEGyAMPH treatment—FIG. 13A) of HFD fed mice treated with PEGyAMPH. This elevation of peripheral adrenergic stimulation was associated with the presence of significantly higher levels of lipolytic markers in circulation, namely Free Fatty Acids (FFAs: PBS—0.851±0.024 μmol/mL in fed-state, 2 h post-injection without access to food; AMPH—0.766±0.043 μmol/mL; PEGyAMPH—1.576±0.326 μmol/mL; p<0.05 for PBS vs PEGyAMPH treatment—FIG. 12B) and Glycerol (PBS—7.399±0.772 μmol/mL in fed-state, 2 h post-injection without access to food; AMPH—11.771±1.249 μmol/mL; PEGyAMPH—19.522±5.991 μg/mL; p<0.05—FIG. 12C). Moreover, there was a marked reduction in iWAT adipocyte size (PBS—4055.0±279.3 μm²; AMPH—1152.0±58.9 μm²; PEGyAMPH—1579.0±49.9 μm²; p<0.0001 for PBS vs AMPH, p<0.01 for PBS vs PEGyAMPH—FIGS. 12D-E) compared to the PBS treated group exposed to the same diet, and a reduction in TGs content both in the liver (PBS—15.50±1.39 μmol/mg of total protein; AMPH—16.23±1.95 μmol/mg; PEGyAMPH—11.49±1.16 μmol/mg; p<0.05 for PBS vs PEGyAMPH treatment), and muscle (PBS—11.77±0.36 μmol/mg of total protein; AMPH—11.54±0.17 μmol/mg; PEGyAMPH—5.92±0.84 μmol/mg; p<0.0001 for PBS vs PEGyAMPH treatment) of PEGyAMPH treated mice which inversely correlates with NE content in such tissues (FIG. 13A). We also evaluated the levels of lipolysis-associated genes during PEGyAMPH treatment and confirmed upregulation in both white and brown adipose tissues (FIGS. 12F-G) as well as the muscle (FIG. 13B), after 10 weeks of HFD exposure. It is also important to report that quantification of gene expression shows that both treatments also altered liver lipid metabolism. Hence, our results show that PEGyAMPH's reduction of weight gain during DIO was associated with a general elevation of the breakdown of peripheral lipid stores.

### PEGyAMPH Treatment Elevates Thermogenesis During DIO.

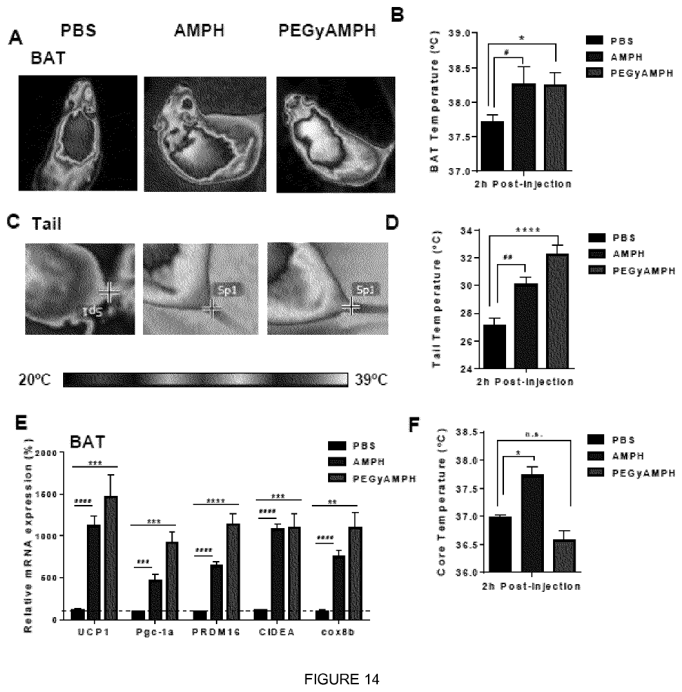

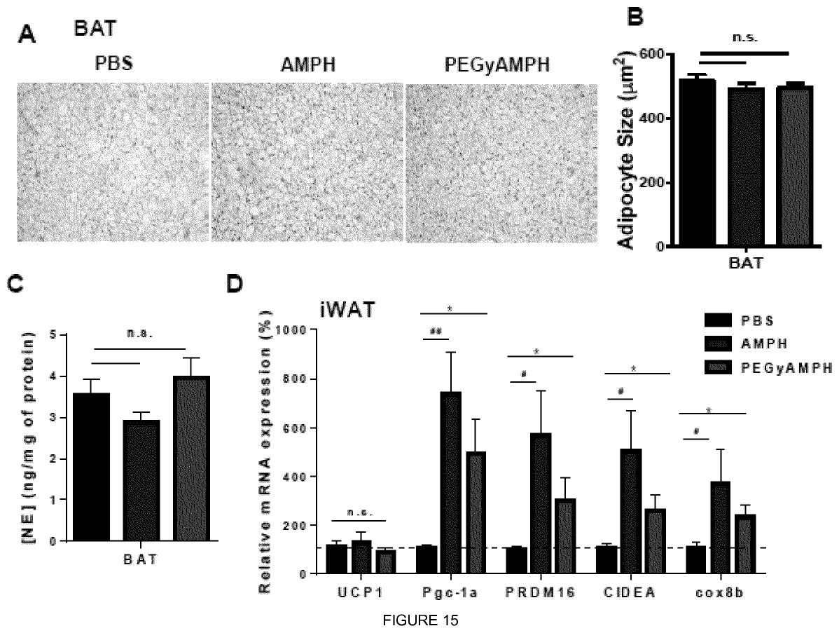

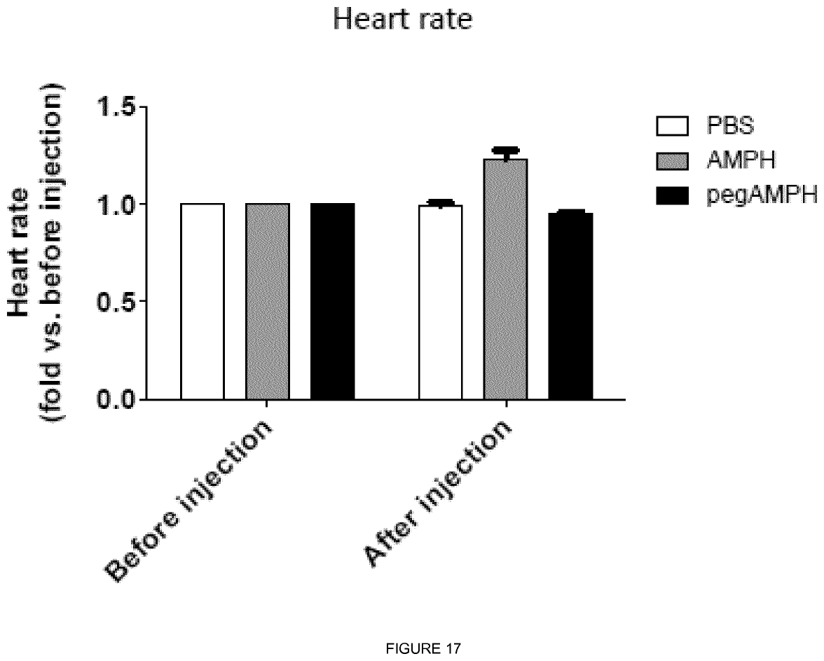

Activation of thermogenesis acts as an energy sink⁵³ and using thermographic photography we detected elevation of BAT temperature after PEGyAMPH treatment in HFD fed mice, 2 h post-injection. This elevation was similar to that evoked by AMPH, compared to control levels (PBS—37.71±0.10° C.; AMPH—38.25±0.25° C.; PEGyAMPH—38.23±0.20° C., p<0.05—FIG. 14A-B). Accordingly, after 10 weeks of HFD and drug treatment, both amphetamines caused a very marked upregulation of BAT UCP1 as well as other thermogenic genes (FIG. 14E). And, although UCP1 levels were not changed in iWAT, all other thermogenic genes quantified were upregulated relative to the levels observed in the control group (FIG. 15D). These results point to a general trend for browning and beginning of adipose tissue after PEGyAMPH treatment, which add onto the upregulation of lipolysis to protect against DIO. Notably, although both drugs act as sympathomimetics, only AMPH caused transient hyperthermia after its administration, as PEGyAMPH treated mice were normothermic as they had similar core body temperature to that of the control group (PBS—37.34±0.14° C.; AMPH—37.94±0.10° C.; PEGyAMPH—37.06±0.27° C., p<0.05 for PBS vs AMPH—FIG. 14F). This suggested that both drugs had differential effects on peripheral heat dissipation. We then probed the levels of heat dissipation by performing thermography at the tail base, and found that PEGyAMPH injected mice had significantly warmer tails relative to the PBS controls (PBS—27.07±0.52° C.; AMPH—30.07±0.54° C.; PEGyAMPH—32.26±0.66° C., p<0.01 for PBS vs AMPH; p<0.0001 for PBS vs PEGyAMPH—FIG. 14C, 14D). As tail temperature is a surrogate measure for peripheral vasodilation, these results indicate that. unlike AMPH which and caused hyperthermia, PEGyAMPH sympathomimetic activity increases thermogenesis without causing vasoconstriction, and mice are still able to maintain normothermia as the heat is dissipated at the extremities. In our models, there were no obvious morphologic differences observed by histologic analysis of BAT between the different groups (FIG. 15A, 15B). PEGyAMPH treatment created a trend towards increased NE in BAT, although with low statistical power (FIG. 15C). These results reveal that PEGyAMPH treatment protects mice against obesity by elevating both lipolysis and thermogenesis, as well as heat dissipation at the extremities. The detrimental cardiac effects of sympathomimetic drugs such as AMPH are believed to originate from an action in the brain; in contrast, pegAMPH was observed to exert a cardioprotective effect (FIG. 17).

Here, we identify a previously undescribed population of sympathetic neuron-associated macrophages (SAMs) that import and degrade NE via specific proteins that are absent from ATMs. We found by transcriptional profiling of isolated SAMs that neural- and adrenergic-related genes are differentially expressed in these cells relative to other macrophage populations. SAMs accumulate intracellular NE despite lacking NE biosynthetic enzymes. Using optogenetics, we demonstrate that SNS activity increases NE content and the pro-inflammatory state of SAMs. We functionally demonstrate that SAMs import and degrade NE via, respectively, an NE transporter (Slc6a2) and a degradation enzyme (monoamine oxidase; MAOa). We further demonstrate that SAM-mediated clearance of extracellular NE contributes to obesity, as inhibiting NE import by SAMs ameliorates obesity, thermogenesis, and browning in ob/ob and high fat diet (HFD)-fed mice. We demonstrate human relevance, as we found that SAMs are also present in human sympathetic ganglia and express similar molecular machinery as mice. Thus, the identification of SAMs provides a novel contribution to the ongoing controversy surrounding the role of macrophages in thermogenesis and obesity while constituting an unforeseen immunological player in noradrenergic homeostasis with therapeutic potential for obesity.

The anti-obesity effect of the loss of function of Slc6a2 from the hematopoietic compartment led to the identification of new mechanism by which Slc6a2 inhibitors, such as amphetamine, promote weight loss and fat mass reduction independently of an action in the brain. It is widely accepted that the primary mechanism of action underlying the anti-obesity effect of AMPH-based drugs is based on its pronounced behavioural effects. However, studies in rodents have suggested that the anti-obesity effects of AMPH and other “anorexigenic” drugs are partly, or even entirely, due to non-behavioural factors^{54, 55, 56}. In that regard, we have herein used genetic sympathectomy to shown that, in conditions of reduced sympathetic tone, diet and exercise are not as effective in controlling body weight. Whereas it is unquestionable that anorexia reduces body weight, our results indicate that this effect depends an intact sympathetic brain-organ axis. AMPHs are small molecules that preferentially accumulate in the brain, and have a short systemic half-life in rodents. Thus its classical sympathomimetic effect may likely be generated centrally, rather than by directly activating SNS neurons peripherally—a conjectured capacity that had not hitherto been reported and that we document herein. To transform a central sympathomimetic into a peripheral one, we had to simultaneously prevent AMPH's access into the brain while extending its peripheral half-life. Pegylation is widely used as a stabilizer that extends the half-life of compounds in circulation, but whether it prevented BBB permeability could not be expected based on literature reporting variable permeability, depending on which molecule is modified. Using mass spectrometry of brain extracts we document that pegylated amphetamine does not cross the BBB, yet it retains its ability to directly activate sympathetic neurons in vitro and in vivo, thus constituting the first peripheral sympathomimetic with a systemic posology and anti-obesity action. PEGyAMPH reduces obesity with a size effect comparable to that of AMPH, yet through a different mechanism of action that spares effects relating to brain penetrance, such as anorexia, hyperkinesia, tremor, and likely addiction or abuse. PEGyAMPH contributes to energy dissipation by activating lipolysis and thermogenesis, which are well known to be driven by elevation of SNS tone both to the WAT and the BAT⁵⁷⁻⁶¹. Moreover, PEGyAMPH may also likely block Slc6a2 expressed by sympathetic associated macrophages that contribute to obesity by taking up and metabolizing norepinephrine^{62,63,64.} AMPH-like compounds such as phentermine are currently approved for short term prescription as anti-obesity agents but are not indicated for long term use due to side effects such as addiction and tacquicardia¹¹. Overall, our results put forward peripheral sympathomimetics as a new generation of anti-obesity compounds and provide candidate compounds for use in promoting weight loss and treating obesity, as described above

TABLE 1

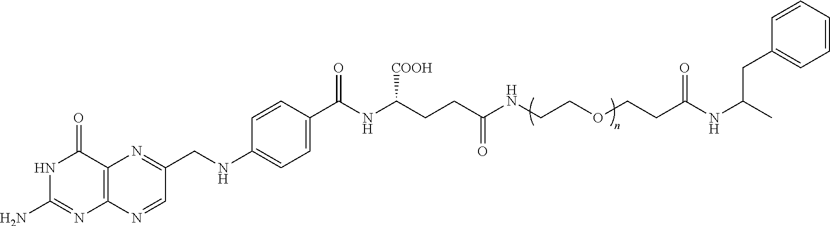

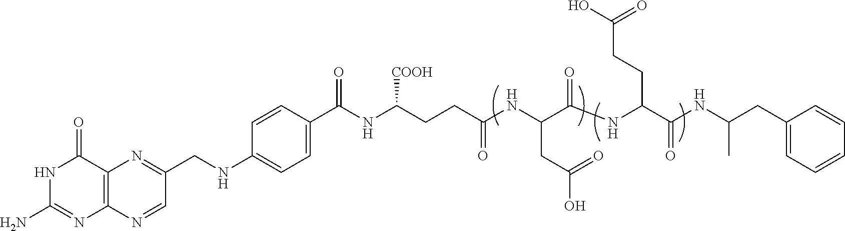

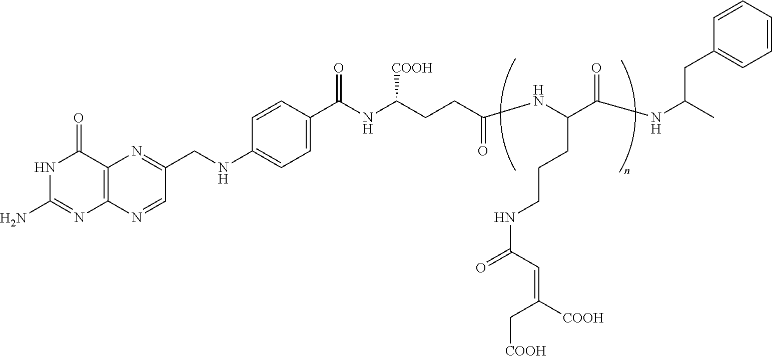

TABLE 2

| | GAPDH (Ct) | Slc6α2 (Ct) | RQ | | GAPDH (Ct) | MAOα (Ct) | RQ |
|---|---|---|---|---|---|---|---|
| SpM | 18.26 | 33.58 | 0.002 | SpM | 19.13 | 32.69 | 0.008 |
| | 23.38 | 33.42 | 0.095 | | 24.09 | 33.32 | 0.167 |
| | 23.79 | 31.98 | 0.343 | | 22.41 | 32.33 | 0.103 |
| | 22.19 | 31.90 | 0.119 | | 18.34 | 29.07 | 0.059 |
| vATM | 20.68 | 34.33 | 0.008 | vATM | 21.68 | 33.08 | 0.037 |
| | 22.65 | 33.53 | 0.053 | | 17.65 | 26.42 | 0.229 |
| | 22.58 | 30.65 | 0.373 | | 20.12 | 28.55 | 0.289 |
| | 22.41 | 33.30 | 0.053 | | 20.46 | 28.52 | 0.374 |
| sATM | 23.21 | 33.49 | 0.080 | sATM | 21.84 | 33.13 | 0.040 |
| | 22.74 | 32.55 | 0.112 | | 24.30 | 32.97 | 0.246 |
| | 24.20 | 33.42 | 0.167 | | 25.86 | 33.80 | 0.405 |
| | 22.93 | 32.29 | 0.152 | | 21.63 | 30.92 | 0.160 |
| SAM ganglia | 30.73 | 33.65 | 13.205 | SAM ganglia | 26.04 | 29.53 | 8.909 |
| | 24.69 | 30.11 | 2.330 | | 26.74 | 31.20 | 4.544 |
| | 27.35 | 31.35 | 6.228 | | 24.16 | 28.79 | 4.039 |
| | 30.54 | 34.11 | 8.448 | | 25.48 | 29.69 | 5.419 |
| | 24.79 | 30.79 | 1.560 | | 25.19 | 30.36 | 2.777 |
| SAM fibers | 28.51 | 32.92 | 4.691 | SAM fibers | 30.01 | 34.00 | 6.296 |
| | 27.17 | 31.72 | 4.267 | | 29.75 | 33.57 | 7.064 |
| | 27.17 | 31.45 | 5.129 | | 30.68 | 33.55 | 13.652 |
| | 29.88 | 33.05 | 11.113 | | 26.10 | 31.53 | 2.317 |
| | 26.77 | 32.05 | 2.584 | | 28.76 | 33.40 | 4.026 |
| Microglia | 23.38 | 33.82 | 0.072 | Microglia | 25.60 | 33.67 | 0.373 |
| | 26.66 | 31.59 | 3.288 | | 24.27 | 34.53 | 0.082 |
| | 24.73 | 33.38 | 0.249 | | 23.77 | 32.04 | 0.325 |
| | 23.62 | 34.11 | 0.070 | | | | |

### REFERENCES

1. Zeng, W. et al. Cell 163, 84-94 (2015).
2. Mathis, D. Cell Metab. 17, 851-859 (2013).
3. Nguyen, K. D. et al. Nature 480, 104-108 (2011).
4. Fischer, K. et al. Nat. Med. 23, 623-630 (2017).
5. Spadaro, O. et al. Cell Rep. 19, 225-234 (2017).
6. Reitman, M. L. Cell Metab. 26, 14-16 (2017).
7. Cooke, D. & Bloom, S. T Nat. Rev. Drug Discov. 5, 919-931 (2006).
8. Melnikova, I. & Wages, D. Nat. Rev. Drug Discov. 5, 369-370 (2006).
9. Riffee, W. H., et al. J. Pharmacol. Exp. Ther. 206, 586-594 (1978).
10. Fuller, R. W., Molloy, B. B., Roush, B. W. & Hauser, K. M. Biochem. Pharmacol. 21, 1299-1307 (1972).
11. Heal, D. J., Smith, S. L., Gosden, J. & Nutt, D. J. J. Psychopharmacol. (Oxf.) 27, 479-496 (2013).
12. Gosselin, D. et al. Cell 159, 1327-1340 (2014).
13. Anlauf, E. & Derouiche, A Front. Endocrinol. 4, (2013).
14. Bignami, A., Eng, L. F., Dahl, D. & Uyeda, C. T. Brain Res. 43, 429-435 (1972).
15. Chaudhry, F. A. et al. Neuron 15, 711-720 (1995).
16. Jessen, K. R. & Mirsky, R. Nat. Rev. Neurosci. 6, 671-682 (2005).
17. Ludwin, S. K., Kosek, J. C. & Eng, L. F. J. Comp. Neurol. 165, 197-207 (1976).
18. Mearow, K. M., Mill, J. F. & Vitkovic, L. Brain Res. Mol. Brain Res. 6, 223-232 (1989).
19. Raff, M. C. et al. Nature 274, 813-816 (1978).
20. Regan, M. R. et al. J. Neurosci. Off. J. Soc. Neurosci. 27, 6607-6619 (2007).
21. Rusnakova, V. et al. PloS One 8, e69734 (2013).
22. Sensenbrenner, M., Lucas, M. & Deloulme, J. C. J. Mol. Med. Berl. Ger. 75, 653-663 (1997).
23. Buttgereit, A. et al. Nat. Immunol. 17, 1397-1406 (2016).
24. Wentworth, J. M. et al. Diabetes 59, 1648-1656 (2010).
25. Amano, S. U. et al Cell Metab. 19, 162-171 (2014).
26. Wolf, Y. et al. Nat. Immunol. 18, 665-674 (2017).
27. Gosselin, D. et al. Science 356, (2017).
28. Clausen, B. E., Burkhardt, C., Reith, W., Renkawitz, R. & Förster, I. Transgenic Res. 8, 265-277 (1999).
29. Merad, M. et al. Nat. Immunol. 3, 1135-1141 (2002).
30. Shirey-Rice, J. K. et al. Dis. Model. Mech. 6, 1001-1011 (2013).
31. Aderem, A. & Underhill, D. M. Annu. Rev. Immunol. 17, 593-623 (1999).
32. Stjärne, L. Rev. Physiol. Biochem. Pharmacol. 112, 1-137 (1989).
33. Schroeder, C. & Jordan, J. Am. J. Physiol. Heart Circ. Physiol. 303, H1273-1282 (2012).
34. Filiano, A. J. et al. Nature 535, 425-429 (2016).
35. Galle-Treger, L. et al. Nat. Commun. 7, (2016).
36. Ibiza, S. et al. Nature 535, 440-443 (2016).
37. Kipnis, J. Science 353, 766-771 (2016).
38. Louveau, A. et al. Nature 523, 337-341 (2015).
39. Rosas-Ballina, M. et al. Science 334, 98-101 (2011).
40. Gautier, E. L. et al. Nat. Immunol. 13, 1118-1128 (2012).
41. Okabe, Y. & Medzhitov, R. Cell 157, 832-844 (2014).
42. Crotti, A. & Ransohoff, R. M. Immunity 44, 505-515 (2016).
43. Prinz, M. & Priller, J. Nat. Rev. Neurosci. 15, 300-312 (2014).
44. Kong, Y. et al. J. Neurosci. Off. J. Soc. Neurosci. 30, 11848-11857 (2010).
45. Neuroglia. (Oxford University Press, 2013).
46. Butovsky, O. et al. Nat. Neurosci. 17, 131-143 (2014).
47. Hanani, M. Brain Res. Brain Res. Rev. 48, 457-476 (2005).
48. Hanani, M. Brain Res. Rev. 64, 304-327 (2010).
49. Gabanyi, I. et al. Cell 164, 378-391 (2016).
50. Camell, C. D. et al. Nature advance online publication, (2017).
51. Pereira, M. M. A. et al. Nat. Commun. 8,14967 (2017).
52. Goodman & Gilman's Pharmacological basis of therapeutics. Chapter 12: Adrenergic Agonists and Antagonists, (McGraw-Hill, 2011).
53. Rothwell, N. J. & Stock, M. J. Nature 281, 31-35 (1979).
54. Arch, J. R. S. Am. J. Clin. Nutr. 34, 2763-2769 (1981).
55. Herling, A. W., Kilp, S., Elvert, R., Haschke, G. & Kramer, W. Endocrinology 149, 2557-2566 (2008).
56. Arch, J. R. S. & Trayhurn, P. Front. Physiol. 4, (2013).
57. Zeng, W. et al. Cell 163, 84-94 (2015).
58. Bartness, T. J., Liu, Y., Shrestha, Y. B. & Ryu, V. Front. Neuroendocrinol. 35, 473-493 (2014).
59. Hausberg, M. et al. Diabetes 51, 2434-2440 (2002).
60. Mahú, I. & Domingos, A. I. Exp. Cell Res. 360, 27-30 (2017).
61. Contreras, C. et al Redox Biol. 12, 854-863 (2017).
62. Camell, C. D. et al. Nature 550, 119-123 (2017).
63. Ravussin, Y. et al. Cell Metab. 28, 289-299.e5 (2018).
64. Pirzgalska, R. M. et al. Nat. Med. 23, 1309-1318 (2017).

1. A conjugate comprising a solute carrier family 6 member 2 (Slc6a2) inhibitor and a moiety which blocks passage across the blood-brain barrier (BBB).

2. A conjugate according to claim 1 wherein the Slc6a2 inhibitor is amphetamine.

3. A conjugate according to claim 1 or claim 2 wherein the BBB blocking moiety comprises a polyether or a, peptide.

4. A conjugate according to claim 3 wherein the BBB blocking moiety is or comprises polyalkyene oxide.

5. A conjugate according to claim 3 wherein the BBB blocking moiety is or comprises polyethylene glycol (PEG) or polypropylene glycol, such as polyethylene glycol (PEG).

6. A conjugate according to claim 5 wherein said PEG moiety comprises 4 or more ethylene oxide units, such as 8 or more ethylene oxide units.

7. A conjugate according to claim 3, wherein the peptide comprises 4 or more amino acid residues, such as 8 or more amino acid residues.

8. A conjugate according to claim 7 wherein the BBB blocking moiety is or comprises a peptide having one or more charged amino acid residues.

9. A conjugate according to claim 8 wherein said one or more charged amino acid residues comprise glutamic acid residues and/or aspartic acid residues

10. A conjugate according to any one of claims 1 to 9 further comprising a targeting moiety.

11. A conjugate according to claim 10 wherein the targeting moiety targets the conjugate to macrophages and/or adipose tissue.

12. A conjugate according to claim 10 wherein the targeting moiety increases the binding of the conjugate to macrophages and/or adipose tissue.

13. A conjugate according to any one of claims 10 to 12 wherein the targeting moiety is a folate group.

14. A conjugate according to any one of claims 10 to 12 wherein the targeting moiety is an antibody molecule.

15. A conjugate according to any one of claims 1 to 13 having a formula set out in Table 1.

16. A conjugate for use according to any one of the preceding claims for use as a medicament.

17. A pharmaceutical composition comprising a conjugate according to any one of claims 1 to 15 and a pharmaceutically acceptable diluent.

18. A method of decreasing fat mass or promoting weight loss comprising administering a Slc6a2 inhibitor that does not cross the BBB to an individual in need thereof.

19. A method of treating obesity comprising administering a Slc6a2 inhibitor that does not cross the BBB to an individual in need thereof.

20. A method according to claim 19 wherein the obesity is diet-induced obesity.

21. A method according to any one of claims 18 to 20 wherein administration of the Slc6a2 inhibitor does not cause hypophagia or hyperkinesia in the individual.

22. A method according to any one of claims 18 to 21 wherein the Slc6a2 inhibitor is a conjugate according to any one of claims 1 to 15.

23. A Slc6a2 inhibitor that does not cross the BBB for use in a method of treatment according to any one of claims 18 to 22.

24. Use of a Slc6a2 inhibitor that does not cross the BBB in the manufacture of a medicament for use in a method of treatment according to any one of claims 18 to 22.
